(19) United States
(12) Patent Application Publication
(10) Pub. No.: US 2025/0290045 A1
(43) Pub. Date: Sep. 18, 2025

(54) SMALL MOLECULES AND METHODS FOR MODULATING TEMPORAL CONTROL OF GELATION AND HYDROGEL MECHANICS

(71) Applicant: The Board of Trustees of the Leland Stanford Junior University, Stanford, CA (US)

(72) Inventors: Sarah C. Heilshorn, Mountain View, CA (US); Sarah Hull, Stanford, CA (US); Aidan Gilchrist, Redwood City, CA (US); Yueming Liu, Redwood City, CA (US); Christopher Lindsay, San Mateo, CA (US); Bauer Lesavage, Redwood City, CA (US); Yan Xia, Stanford, CA (US); Junzhe Lou, Redwood City, CA (US)

(21) Appl. No.: 18/862,480

(22) PCT Filed: May 5, 2023
(86) PCT No.: PCT/US2023/066679

§ 371 Date: Nov. 1, 2024

Related U.S. Application Data
(60) Provisional application No. 63/338,648, filed on May 5, 2022.

(51) Int. Cl. C12N 5/071; B01J 31/02; C08L 5/08; C08L 89/00

(52) U.S. Cl. CPC C12N 5/0671; B01J 31/0271; C08L 5/08; C08L 89/00; C12N 5/0672; C12N 2513/00

(57) ABSTRACT

Methods and compositions for modulating hydrogel gelation and mechanics for 3D cell culture are provided. Hydrogels have a variety of benefits for cell and tissue culture, including without limitation homogeneous distribution of materials and cells at the initiation of the 3D cell culture, disruption of network formation and disassociation of the 3D cell culture for cell retrieval, etc. The method for modulating the stiffness of a hydrogel solution comprises contacting a hydrogel solution with an effective amount of a competitor agent sufficient to modulate the stiffness of the hydrogel solution. In some embodiments, the methods further comprise contacting the hydrogel solution with a catalyst agent wherein the catalyst agent increases the forward and reverse reaction rate of reversible covalent bond formation that occurs in the hydrogel solution.

### CROSS-REFERENCE TO RELATED APPLICATIONS

Pursuant to 35 U.S.C. § 119 (e), this application claims priority to the filing date of U.S. Provisional Patent Application Ser. No. 63/338,648 filed May 5, 2022, the disclosure of which application is herein incorporated by reference.

### GOVERNMENT RIGHTS

This invention was made with Government support under contract 2033302 awarded by the National Science Foundation. The Government has certain rights in the invention.

### SEQUENCE LISTING

A Sequence Listing is provided herewith as a Sequence Listing XML, “STAN-1970WO Sequence Listing” created May 4, 2023 and having a size of 36,615 bytes. The contents of the txt file Sequence Listing XML are incorporated by reference herein in their entirety.

### BACKGROUND

Current methods for 3-dimensional (3D) cell culture often rely upon animal-derived proteins that possess inherent batch-to-batch variability, xenogenic factors, and have limited potential for clinical translation. Additionally, retrieval of cells from the culture system is required for continued culture and analysis of cells. However, enzymatic methods that are used to degrade the hydrogel result in non-specific cleavage and downstream effects, resulting in altered geno- and phenotypic profiles. Variability and complexity of animal-sourced materials and harsh cell retrieval methods limit the interpretation and reproducibility of cell-based studies. Moreover, current hydrogel systems are limited by the rate of gelation, where rapid gelation can result in inadequate mixing and a heterogeneous distribution of materials and cells. To advance 3D cell culture there is a clear need for a chemically-defined, non-animal, xenogen-free culture system with control over gelation and degradation properties.

Provided herein are methods and compositions for the reversible modulation of 3D cell culture hydrogel gelation and mechanics.

### SUMMARY

Methods and compositions for modulating gelation and mechanics in hydrogel solutions used for 3D cell culture are described, which have a variety of benefits for cell and tissue culture, including without limitation homogeneous distribution of materials and cells at the initiation of the 3D cell culture, disruption of network formation and disassociation of the 3D cell culture for cell retrieval, etc. The methods utilize (1) polymers modified with functional groups that can participate in reversible covalent bond formation, (2) a small molecule competitor agent with a functional group that competes for binding sites with the polymer, and/or (3) a small molecule catalyst that increases the forward and reverse reaction rate of the reversible covalent bond formation.

In some embodiments, a method for modulation of a hydrogel solution comprises contacting a hydrogel solution with an effective amount of a competitor agent sufficient to modulate the stiffness of the hydrogel solution. The competitor agent disrupts bond formation of the polymeric hydrogel, softening the material due to the decreased number of available crosslinking sites. The addition of the competitor agent during gel formation increases the time of gelation, allowing for improved mixing, and homogeneous distribution of material and cells within the hydrogel. After the addition of cell culture media to the hydrogel, the competitor agent can unbind and diffuse away from the hydrogel, gradually stiffening the hydrogel over time. Alternatively, the competitor can be added at a later time point to soften the hydrogel over time or to completely disrupt network formation and disassociate the hydrogel, allowing for easy cell retrieval for downstream analysis.

In some embodiments, the methods further comprise contacting the hydrogel solution with a catalyst agent wherein the catalyst agent alters the forward and reverse reaction rate of reversible covalent bond formation that occurs in the hydrogel solution. The catalyst agent can be added to increase bond exchange rate, which in turn can alter a cell's ability to remodel its extracellular environment, providing in vitro models that recapitulate features of the native tissue environment.

The hydrogel solution comprises a mixture of a first polymer and a second polymer, wherein the first polymer is modified with a first functional group and the second polymer is modified with a second functional group, wherein the first functional group of the first polymer can participate in reversible covalent bond formation with the second functional group of the second polymer. In some embodiments, the first polymer is chemically modified hyaluronic acid (HA) and the second polymer is chemically modified elastin-like protein (ELP). In some embodiments, the first polymer is chemically modified with the first functional group and the second polymer is chemically modified with the second functional group. In some embodiments, the first functional group comprises an aldehyde or benzaldehyde-containing functional group and the second functional group comprises a hydrazine-containing functional group. In some embodiments, the first functional group comprises a hydrazine-containing functional group and the second functional group comprises an aldehyde or benzaldehyde-containing functional group. The mixture of these two components within the hydrogel solution results in the formation of hydrazone bonds resulting in a hydrogel network, referred to herein as “HELP”.

The competitor agent can be any agent that comprises a functional group that is analogous to the first or second functional group of the first or the second polymer. In some embodiments, the competitor agent comprises an aldehyde, a benzaldehyde or a hydrazine functional group. In some embodiments, the competitor agent is selected from the group of 2-hydrazinoethanol, benzaldehyde, 4-hydrazinobenzoic acid, hydrazinoacetic acid, butyraldehyde, and derivatives thereof. An effective amount of the competitor agent for use in the present disclosure can be any amount of the competitor agent sufficient to modulate the stiffness of the hydrogel solution. For instance, the effective amount of the competitor agent may be at a concentration from least about 0.001-1 mM, 1-5 mM, 5-10 mM, 10-15 mM, 15-20 mM, 20-25 mM or greater than about 25 mM.

In some embodiments, a method for modulating the stiffness of the hydrogel solution comprises contacting the hydrogel solution with a catalyst agent. The catalyst agent can be any agent that increases the forward and reverse reaction rate of the reversible covalent bond formation in the hydrogel solution. In some embodiments, the catalyst agent is selected from the group of 2-(Aminomethyl)benzimidazole, 2-(aminomethyl) imidazoles, N,N-dimethylethylenediamines, 2-aminobenzenephosphonic acids, 2-aminophenols and derivatives thereof. An effective amount of the catalyst agent for use in the present disclosure can be any amount of the catalyst agent sufficient to modulate the stiffness of the hydrogel solution. For instance, the effective amount of the catalyst agent may be at a concentration from at least about 0.1-5 mM, 5-10 mM, 10-15 mM, 15-20 mM, 20-100 mM or greater than about 100 mM. In embodiments where the hydrogel solution is contacted with the competitor agent and the catalyst agent, they may be provided in a specific ratio to one another. For instance, the competitor agent and the catalyst agent may be in a ratio of about 1:1 to about 1:100 or in a ratio 100:1 to about 1:1.

In some embodiments, an effective amount of a competitor agent reduces the stiffness of the hydrogel solution by at least about 2 fold, 3 fold, 4 fold, 5 fold, 6 fold, 7 fold, 8 fold, 9 fold, 10 fold or greater than about 10 fold. In some embodiments, the effective amount of the competitor agent and the catalyst agent reduces the stiffness of the hydrogel solution by at least about 2 fold, 3 fold, 4 fold, 5 fold, 6 fold, 7 fold, 8 fold, 9 fold, 10 fold or greater than about 10 fold.

Also provided herein are methods for increasing the gelation time of a hydrogel solution. The methods comprise contacting the hydrogel solution with an effective amount of a competitor agent sufficient to alter the gelation time of the hydrogel solution. In some embodiments, the effective amount of a competitor agent increases the gelation time by at least about 10%, 20%, 30%, 40%, 50%, 60%, 70%, 80%, 90%, 100% or greater than about 100% increase in the gelation time relative to the absence of the competitor agent.

Also provided herein are methods for decreasing the gelation time of a hydrogel solution. The methods comprise contacting a hydrogel solution with an effective amount of a catalyst agent sufficient to decrease the gelation time of a hydrogel solution. The effective amount of a catalyst agent is any amount of a catalyst agent sufficient to decrease the gelation time of a hydrogel solution. For instance, the effective amount of the catalyst agent may be at a concentration from least about 0.1-5 mM, 5-10 mM, 10-15 mM, 15-20 mM, 20-100 mM or greater than about 100 mM. In some embodiments, the effective amount of the catalyst agent decreases the gelation time by at least about 10%, 20%, 30%, 40%, 50%, 60%, 70%, 80%, 90%, 100% or greater than about 100% decrease in the gelation time relative to the absence of the catalyst agent.

In another aspect of the invention, a method is provided for the retrieval of cells from a 3D cell culture, the method comprising contacting the 3D cell culture with an effective amount of a competitor agent sufficient to dissociate the 3D cell culture wherein the 3D cell culture comprises a hydrogel solution comprising a mixture of a first polymer and a second polymer wherein the first polymer is a modified with a first functional group and the second polymer is modified with a second functional group wherein the first functional group of the first polymer can participate in reversible covalent bond formation with the second functional group of the second polymer. In some embodiments, the first polymer is chemically modified hyaluronic acid (HA) and the second polymer is chemically modified elastin-like protein (ELP). In some embodiments, the method further comprises contacting the 3D cell culture with an effective amount of a catalyst agent sufficient to dissociate the 3D cell culture.

In another aspect of the invention, kits are provided comprising the hydrogel solution and the competitor agent. In some embodiments, the kit further comprises the catalyst agent.

### BRIEF DESCRIPTION OF THE DRAWINGS

FIG. 1 is a schematic depiction of exemplary methods of the invention.

FIG. 2 depicts the effect of hydrazinoacetic acid on the storage modulus of a HELP hydrogel.

FIG. 3 depicts the effect of different concentrations of hydrazinoacetic acid on the storage modulus of a HELP hydrogel over time.

FIG. 4 depicts the effect of different concentrations of hydrazinoacetic acid on the gelation time of a HELP hydrogel.

FIG. 5 depicts exemplary competitor agents.

FIG. 6 depicts the effect of competitor agent on the fold change in stiffness of HELP hydrogels relative to absence of competitor.

FIG. 7 depicts the forward and reverse reaction rates and association constants of two exemplary competitor agents.

FIG. 8 depicts the effect of diffusion of two exemplary competitor agents on the fold change of the stiffness of HELP hydrogels over time.

FIG. 9 depicts the effect of altering the ratio of catalyst to competitor on the storage modulus of HELP hydrogels.

FIG. 10 Library of competitors for transient disruption of hydrazone crosslinks. A. The reaction of aldehyde and hydrazine to form hydrazone and water is a dynamic, reversible reaction that occurs at physiological conditions. B. Schematic of a transient competitor for modulating crosslinking density. Small molecule competitors can diffuse into the hydrogel to temporarily decrease crosslinking density or diffuse out of the hydrogel to allow crosslinks to form. Crosslinking sites are circled with dotted lines. C. Schematic of HELP matrix. HELP is composed of benzaldehyde-modified hyaluronan (HA-BZA) and hydrazine-modified elastin-like protein (ELP-HYD). When mixed, these two components form a HA-ELP (HELP) hydrogel (SEQ ID NO: 28). D. The library of competitors includes small molecules with either aldehyde or hydrazine functional groups that can react with ELP-HYD or HA-BZA, respectively. Each group contains a competitor with an alkyl or aromatic side group.

FIG. 11 Competitors decrease the stiffness of HELP hydrogel in a dose-dependent manner. A. Schematic of the two reactions that are simultaneously occurring in a HELP hydrogel with a competitor. The top competitor reaction (K_{eq}¹) occurs between the crosslinking moiety (A) and the unbound competitor (C) to form a blocked group (AC) that cannot participate in crosslinking. The bottom crosslinking reaction (K_{eq}²) occurs between the crosslinking moieties of the two polymers, (A) and (B), to form a crosslink (AB). B. A frequency sweep of HELP hydrogels with 10 mM of competitor shows a decrease in the storage modulus (G) that is dependent upon the competitor chemistry. Each trace represents an average of three independent runs (n=3). C. and D. The addition of either HydAA or Butanal led to a dose-dependent decrease in storage modulus (G) of a HELP hydrogel. Data shown are mean±sd of n=3. Statistical significance was tested by one-way ANOVA with Tukey's multiple comparisons testing: **=p<0.01, ***=p<0.001, ****=p<0.0001. E. Correlation of HELP hydrogel storage modulus and equilibrium ratio of hydrazone. Pearson r values are reported. Dashed line represents linear regression fit for visualization purposes. F. Predicted gel-sol phase diagram of HELP, depending upon relative reactivity of competitor and concentration. Points are competitor concentration ratios that were empirically tested.

FIG. 12 Competitors increase gelation time and decrease hydrogel heterogeneity. A. Competitor hydrazinoacedic acid (HydAA) does not significantly increase the gelation time of HELP hydrogel. B. Competitor butyraldehyde (Butanal) significantly increases gelation time in a dose-dependent manner. Data shown are mean±sd of n=3-4. Statistical significance was tested by one-way ANOVA with Tukey's multiple comparisons testing: **=p<0.01, ****=p<0.0001. D. Predicted gelation time with different competitor concentration. Normalized to gelation time without competitors. E. Representative fluorescent images of HELP with or without 10 mM Butanal. F. Quantification of HELP hydrogel homogeneity. Left: Pixel intensity of images in E. Right: Quantified intensity variance of HELP hydrogel with or without 10 mM butanal. Data shown are mean±sd of n=4. Unpaired two tailed Student's t-test; *=p<0.05, ****=p<0.0001.

FIG. 13 A) Schematic of competitor diffusion model. Assume competitors can only diffuse towards x>0 direction and surrounding media is an infinite sink. B) Modulus recovery over 48 hours of HELP when submerging the hydrogel in fresh PBS. HELP with 10 mM HydAA shows rapid recovery whereas HELP with 10 mM butanal shows slow recovery. One-way ANOVA with Tukey's multiple comparisons testing; *=p<0.05, ns=not significant, n=3. Data shown are mean±sd. C) Cumulative release of 2.8 mM butanal over 21 days. Dots: experimental measurements. Dashed line: fitted curve using one-dimensional diffusion model.

FIG. 14 Competitor is not cytotoxic to cultured cells. A) Hepatic organoids (HO) were cultured as single cells in soft, stiff, and stiff+competitor. Robust formation of HO in the presence of competitor during both the growth and differentiation phase was seen. B) Staining of the cytoskeleton of mature, differentiated HO (Day 16) reveals no aberrant morphology due to the competitor. C) As the competitor diffuses out over several days, with diminishing concentration with time, cytotoxicity during the early phase of cell growth (4-days) was explored when the competitor concentration is highest. Using lactase dehydrogenase, a metric of cytotoxicity, there was no significant difference observed in cytotoxicity across all of the conditions. D) On day 3, organoid formation efficiency was quantified. The presence of the competitor led to an increase in formation efficiency compared to the stiff environment, and resulted in a formation efficiency that was comparable to that of the soft environment. E) shows that over a 16 day culture period, the addition of competitor enabled growth comparable to that of a soft environment.

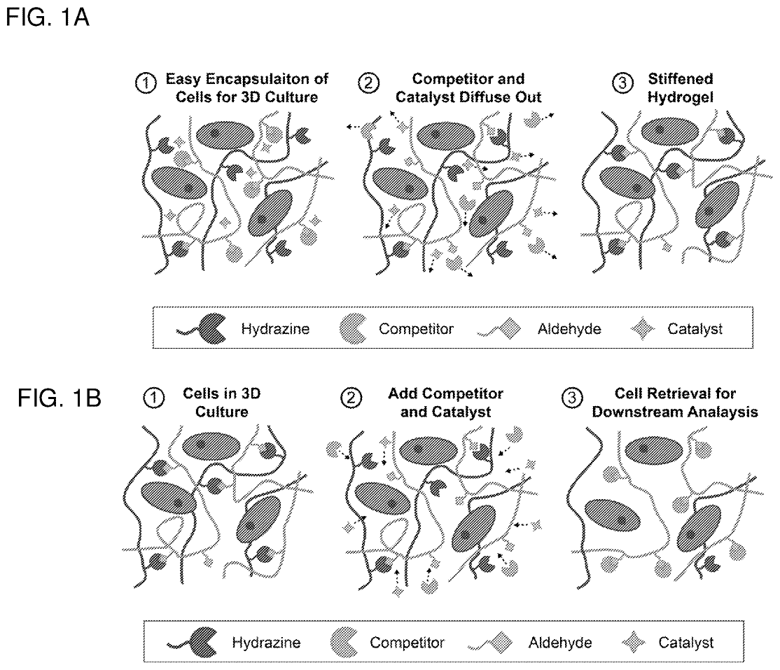

### DETAILED DESCRIPTION

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The term “hydrogel” is used in its conventional sense to refer to a material that absorbs a solvent (e.g. water), undergoes swelling without measurable dissolution, and maintains three-dimensional networks capable of reversible deformation. “Swelling” as referred to herein is meant the isotropic expansion of the hydrogel structure as water molecules diffuse throughout the internal volume of the hydrogel. The properties of copolymer hydrogels disclosed herein may be modulated as desired, by varying the amounts of each component, ratios of each component or the density of specific components, as described in greater detail below. The term hydrogel may include both desiccated and hydrated (e.g., solvent-swollen) hydrogels.

In some embodiments of the invention, the hydrogel provides a scaffold for cell growth, including growth of metabolically active cells, e.g. differentiating cells, etc. The cells may be grown in vitro, e.g. a culture of one or a plurality of cell types. Cells may also be grown in vivo, e.g. where a hydrogel provides a substrate for regenerative cell growth. The hydrogels used in the invention provide appropriate mechanical strength for long-term structural stability.

An Elastin-like Protein (ELP) comprises a recombinant sequence of elastin-like sequences optionally interspersed with cell-adhesive sequences. To engage in crosslinking with chemically modified HA, the ELP is chemically modified to comprise a pendant hydrazine group. The optional cell-adhesive sequence within the ELP comprises a motif involved in cell adhesion, which may be selected from an integrin-binding, fibronectin-based, extended RGD sequence, a scrambled RGD sequence, a cell-adhesive sequence derived from collagen type I, e.g. (SEQ ID NO:3) DGEA, a cell adhesive sequence derived from tenascin, e.g. (SEQ ID NO:4) PLAEIDGIELTY, (SEQ ID NO: 5) VFDNFVLK, etc.; a cell adhesive sequence derived from laminin, e.g. (SEQ ID NO:6) IKVAV, (SEQ ID NO:7) YIGSR, etc.; a cell adhesive sequence derived from cadherin, e.g. (SEQ ID NO: 8) HAVDI, (SEQ ID NO:9) HAVDIHAVDI; and the like. ELPs have previously been described in PCT Application No. PCT/US21/57925, herein specifically incorporated by reference.

The cell-adhesive domain of the engineered elastin-like protein can be designed to include alternative peptide sequences known to interact with cell-surface receptors. These sequences can include peptides derived from native extracellular matrix proteins (e.g. fibronectin, laminin, collagen, tenascin-C) or peptides derived from cell-cell adhesion receptors (e.g. N-cadherin). Selection of the cell adhesive peptide sequence together with the elastin-like region sequence defines the overall hydrophobicity of the engineered protein and hence controls the lower critical solution temperature (LCST) behavior.

In some embodiments, an ELP comprises the structure:

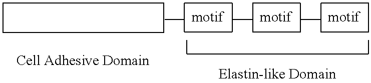

where the cell adhesive domain is from about 15 to about 45 amino acids in length and comprises one or more cell adhesion sequence motifs, which may be selected from RGD, scrambled RGD, no RGD, or any of SEQ ID NO:3 to SEQ ID NO:9. SEQ ID NO: 10-19 and 22 are exemplary.

Linker sequences optionally flank the cell adhesion sequence motif, where a peptide linker can be between about 5 to 20, 5 to 15, 5 to 10 or 5 to 9 amino acids in length. Exemplary linkers include linear peptides having at least two amino acid residues such as Gly-Gly, Gly-Ala-Gly, Gly-Pro-Ala, Gly-Gly-Gly-Gly-Ser (SEQ ID NO:26). Suitable linear peptides include poly glycine, polyserine, polyproline, polyalanine and oligopeptides consisting of alanyl and/or serinyl and/or prolinyl and/or glycyl amino acid residues. In one embodiment a linker comprises the amino acid sequence GSTSGSGKSSEGKG (SEQ ID NO:27).

The elastin-like domain is comprised of elastin-like motifs, which include, without limitation, (SEQ ID NO:23) VPGIG; (SEQ ID NO:24) VPGKG; (SEQ ID NO:25) VPGYG. One or more of SEQ ID NO:23, 24 and 25 can be present in a protein. In some embodiments the number of motifs is from 1 to 7, from 1 to 6, from 2 to 5, from 3 to 5; and may be about 5 motifs. Exemplary domain sequences are provided in, for example, SEQ ID NO:20 and 21. Examples include, without limitation, SEQ ID NO:1, LQ(LDASTVYAVGRGDSPASSA[(VPGIG)₂VPGKG(VPGIG)₂]₃)₄ and SEQ ID NO:2, LQ(LDASTVYAVGRDGSPASSA[(VPGIG)₂VPGKG(VPGIG)₂]₃)₄.

The ELP protein is chemically modified to comprise a pendant hydrazine group, and may comprise from about 3 to about 20 hydrazine groups, from about 5 to about 18, from about 10 to about 14 groups. Standard bioconjugation chemistry can be used to attach pendant hydrazines at sites of any of lysine, cysteine, or tyrosine amino acids.

TABLE 1
Amino-acid sequences for the bio-active domain and elastin-like region of HELP gels.

| ECM-derived | | |
|---|---|---|
| Protein | Cell-adhesive domain | Elastin-Like Region |
| h-Fibronectin | (SEQ ID NO: 10) TVYAVTGRGDSPASSAA | (SEQ ID NO: 20)(VPGIG)₂(VPGKG)(VPGIG)₂ |
| Laminin (ß1 | (SEQ ID NO: 11) VSDPGYIGSRSDDSASASAA | (SEQ ID NO: 20) (VPGIG)₂(VPGKG)(VPGIG)₂ |
| chain) | | |
| Laminin (α1 | (SEQ ID NO: 22) ARKQAASIKVAVSADRASA | (SEQ ID NO: 21) |
| chain) | | (VPGIG)(VPGKG)(VPGYG)(VPGIG)(VPGKG)(VPGIG) |
| Collagen I | (SEQ ID NO: 12) VGPAGGDGEAGAQGPP | (SEQ ID NO: 20) (VPGIG)₂(VPGKG)(VPGIG)₂ |
| h-Tenascin-C | (SEQ ID NO: 13) | (SEQ ID NO: 20) (VPGIG)₂(VPGKG)(VPGIG)₂ |
| | SGSGGSGGPLAEIDGIELTYGGSGGSGS | |
| h-Tenascin-C | (SEQ ID NO: 14) | (SEQ ID NO: 21) |
| | SGSGGSGGLDVFDNFVLKGGSGGSGS | (VPGIG)(VPGKG)(VPGYG)(VPGIG)(VPGKG)(VPGIG) |
| h-Tenascin-C | (SEQ ID NO: 15) | (SEQ ID NO: 20) (VPGIG)₂(VPGKG)(VPGIG)₂ |
| | SGSGGSGGLDVFDNFVLGGSGGSGS | |
| N-cadherin | (SEQ ID NO: 16) SGSGGSGGHAVDIGGSGGSGS | (SEQ ID NO: 20) (VPGIG)₂(VPGKG)(VPGIG)₂ |
| N-cadherin | (SEQ ID NO: 17) | (SEQ ID NO: 20) (VPGIG)₂(VPGKG)(VPGIG)₂ |
| | SGSGGSGGHAVDINGHAVDIGGSGGSGS | |
| Non-binding | (SEQ ID NO: 18) SGSGGSGGADHIVGGSGGSGS | (SEQ ID NO: 20) (VPGIG)₂(VPGKG)(VPGIG)₂ |
| Non-binding | (SEQ ID NO: 19) TVYAVTGRDGSPASSAA | (SEQ ID NO: 20) (VPGIG)₂(VPGKG)(VPGIG)₂ |

Hyaluronic acid is an anionic, non-sulfated glycosaminoglycan distributed widely throughout connective, epithelial, and neural tissues. Hyaluronic acid is a polymer of disaccharides, themselves composed of D-glucuronic acid and N-acetyl-D-glucosamine, linked via alternating β-(1→4) and β-(1→3) glycosidic bonds. Hyaluronic acid can be up to 25,000 disaccharide repeats in length. Polymers of hyaluronic acid can range in size from about 20 kDa to about 1.5 MDa; from about 20 kDa to about 1 MDa.

The hyaluronic acid is chemically modified to comprise a pendant benzaldehyde or aldehyde side group. The HA is usually modified at from about 5% to about 40% of the available reactive groups and may be from about 7% to about 20%, from about 10% to about 15%, from about 20% to about 40% and may be around 12% modified.

For an aldehyde functional group, the carboxylic acid groups on HA are amidated with propargylamine, generating an HA-alkyne intermediate; then, copper click chemistry was used to react this alkyne with the azide moiety of a heterobifunctional small molecule containing an aldehyde functional group onto the HA, generating HA functionalized with aldehydes.

Benzaldehyde modification can be accomplished by first modifying HA to comprise alkyne groups at a desired concentration, e.g. from about 3% to about 40%. HA-alkynes are then modified with an azide moiety of a heterobifunctional small molecule containing a benzaldehyde functional group to generate HA-benzaldehyde.

The term “hydrazone bond” as used herein refers to the bond formed by the following chemical reaction:

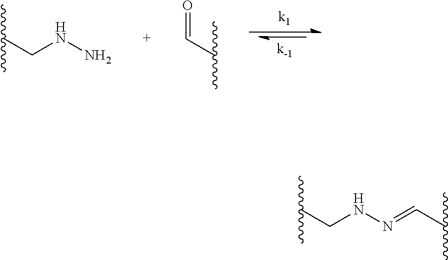

The term “storage modulus” as used herein may be used interchangeably with the term “stiffness”. Storage modulus refers to the ratio of the elastic stress to strain and indicates a material's ability to store energy. The storage modulus may be measured using oscillatory shear rheology

“Competitor agent” as used herein refers to any agent that competes with the first or the second functional group of the first or second polymer within the hydrogel solutions for reversible covalent bond formation. Agents that compete with the first or the second functional group are agents that comprise functional groups that are analogous to the first or the second functional group. For instance, if the first functional group comprises an aldehyde or benzaldehyde-containing functional group and the second functional group comprises a hydrazine-containing functional group then the competitor agent comprises an aldehyde, benzaldehyde or hydrazine-containing functional group.

“Catalyst agent” as used herein refers to any agent that increases the forward and reverse reaction rate of the reversible covalent bond formation in the hydrogel solution. For instance, catalyst agents of the present disclosure increase the forward and reverse reaction rate of the reversible covalent bond formation between the first and second functional group in the first and second polymers of the hydrogel solution. Catalyst agents of the present disclosure also increase the forward and reverse reaction rate of the reversible covalent bond formation between the competitor agent and the first or the second functional group of the first or the second polymer.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction in viral titers).

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; avians, and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like.

The terms “nucleic acid molecule” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “somatic cell” encompasses any cell in an organism that cannot give rise to all types of cells in an organism, i.e. it is not pluripotent. In other words, somatic cells are cells that have differentiated sufficiently that they will not naturally generate cells of all three germ layers of the body, i.e. ectoderm, mesoderm and endoderm.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). A “stem cell” is a cell characterized by the ability of self-renewal through mitotic cell division and the potential to differentiate into a tissue or an organ. Among mammalian stem cells, embryonic and somatic stem cells may be distinguished. Pluripotent stem cells, which include embryonic stem cells, embryonic germ cells and induced pluripotent cells, can contribute to tissues of a prenatal, postnatal or adult organism.

The terms “primary cells”, “primary cell lines”, and “primary cultures” are used interchangeably herein to refer to cells and cell cultures that have been derived from a subject and allowed to grow in vitro for a limited number of passages, i.e. splittings, of the culture. For example primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

The subject cells may be from any mammal, including humans, primates, domestic and farm animals, and zoo, laboratory or pet animals, such as dogs, cats, cattle, horses, sheep, pigs, goats, rabbits, rats, mice etc. They may be established cell lines or they may be primary cells, where “primary cells”, “primary cell lines”, and “primary cultures” are used interchangeably herein to refer to cells and cells cultures that have been derived from a subject and allowed to grow in vitro for a limited number of passages.

The subject cells may be isolated from fresh or frozen cells, which may be from a neonate, a juvenile or an adult, and from tissues including skin, muscle, bone marrow, peripheral blood, umbilical cord blood, spleen, liver, pancreas, lung, intestine, stomach, adipose, and other differentiated tissues. The tissue may be obtained by biopsy or aphoresis from a live donor, or obtained from a dead or dying donor within about 48 hours of death, or freshly frozen tissue, tissue frozen within about 12 hours of death and maintained at below about −20° C., usually at about liquid nitrogen temperature (−190° C.) indefinitely. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

The term “cell culture” or “culture” means the maintenance of cells in an artificial, in vitro environment. It is to be understood, however, that the term “cell culture” is a generic term and may be used to encompass the cultivation not only of individual cells, but also of tissues or organs.

Culture conditions of interest provide an environment permissive for differentiation, in which stem or progenitor cells will proliferate, differentiate, or mature in vitro. Such conditions may also be referred to as differentiative conditions. Features of the environment include the medium in which the cells are cultured, any growth factors or differentiation-inducing factors that may be present, and a supporting structure of a hydrogel as disclosed herein. Differentiation may be initiated by formation of organoids, or similar structures.

A “long term culture” used herein refers to a culture in which cells grow, differentiate and are viable for at least about 10 days, or more than 30 days, or more than 60 days, or more than 100 days or more than 150 days.

The term “explant” is used herein to mean a piece of tissue and the cells thereof originating from mammalian tissue that is cultured in vitro, for example according to the methods of the invention. The mammalian tissue from which the explant is derived may obtained from an individual, i.e. a primary explant, or it may be obtained in vitro, e.g. by differentiation of induced pluripotent stem cells.

The term “organoid” is used herein to mean a 3-dimensional growth of mammalian cells in culture that retains characteristics of the tissue in vivo, e.g. prolonged tissue expansion with proliferation, multilineage differentiation, recapitulation of cellular and tissue ultrastructure, etc. A primary organoid is an organoid that is cultured from an explant, i.e. a cultured explant. A secondary organoid is an organoid that is cultured from a subset of cells of a primary organoid, i.e. the primary organoid is fragmented, e.g. by mechanical or chemical means, and the fragments are replated and cultured. A tertiary organoid is an organoid that is cultured from a secondary organoid, etc.

“Ultrastructure” refers to the three-dimensional structure of a cell or tissue observed in vivo. For example, the ultrastructure of a cell may be its polarity or its morphology in vivo, while the ultrastructure of a tissue would be the arrangement of different cell types relative to one another within a tissue.

### Methods of the Invention

In one aspect, this application is directed to methods for modulating the stiffness of a hydrogel solution, and for culturing cells in such hydrogels. The methods comprise contacting a hydrogel solution with an effective amount of a competitor agent sufficient to modulate the stiffness of the hydrogel solution. In some embodiments, the methods further comprise contacting the hydrogel solution with a catalyst agent wherein the catalyst agent increases the forward and reverse reaction rate of reversible covalent bond formation that occurs in the hydrogel solution.

The hydrogel solution comprises a mixture of a first polymer and a second polymer wherein the first polymer is modified with a first functional group and the second polymer is modified with a second functional group wherein the first functional group of the first polymer can participate in reversible covalent bond formation with the second functional group of the second polymer. In some embodiments, the first polymer is chemically modified hyaluronic acid (HA) and the second polymer is chemically modified elastin-like protein (ELP). In some embodiments, the first polymer is chemically modified with the first functional group and the second polymer is chemically modified with the second functional group. In some embodiments, the first functional group comprises an aldehyde or benzaldehyde-containing functional group and the second functional group comprises a hydrazine-containing functional group. In some embodiments, the first functional group comprises a hydrazine-containing functional group and the second functional group comprises an aldehyde or benzaldehyde-containing functional group. The mixture of these two components within the hydrogel solution results in the formation of hydrazone bonds resulting in a hydrogel network, referred to herein as “HELP”. HELP hydrogels have been previously described, for instance, by PCT Application No. PCT/US21/57925, herein specifically incorporated by reference.

The competitor agent may be any agent that comprises a functional group that is analogous to the first or second functional group of the first or the second polymer. In some embodiments, the competitor agent comprises an aldehyde, a benzaldehyde or a hydrazine functional group. In some embodiments, the competitor agent is selected from the group of 2-hydrazinoethanol, benzaldehyde, 4-hydrazinobenzoic acid, hydrazinoacetic acid, butyraldehyde, and derivatives thereof. An effective amount of the competitor agent for use in the present disclosure may be any amount of the competitor agent sufficient to modulate the stiffness of the hydrogel solution. For instance, the effective amount of the competitor agent may be at a concentration from at least about 0.001-1 mM, 1-5 mM, 5-10 mM, 10-15 mM, 15-20 mM or greater than about 20 mM. The competitor agent may be at any intervening concentration within a specific range. For instance, when the competitor agent is from at least about 1-5 mM, the concentration of the competitor agent may be about 1 mM, about 2 mM, about 3 mM, about 4 mM or about 5 mM.

In some embodiments, the method for modulating the stiffness of the hydrogel solution further comprises contacting the hydrogel solution with a catalyst agent. The catalyst agent may be any agent that increases the forward and reverse reaction rate of the reversible covalent bond formation in the hydrogel solution. In some embodiments, the catalyst agent is selected from the group of 2-(aminomethyl) imidazoles, N,N-dimethylethylenediamines, 2-aminobenzenephosphonic acids, 2-aminophenols and derivatives thereof. Other exemplar catalyst agents have been described by Larson et al. (Chem Sci. 2018 May 21;9 (23):5252-5259), Crisalli et al. (Org Lett. 2013 Apr. 5;15 (7):1646-9) and Larsen et al. (Org Lett. 2015 Jan. 16;17(2):274-7), each herein incorporated by reference.

An effective amount of the catalyst agent for use in the present disclosure may be any amount of the catalyst agent sufficient to modulate the gelation time or mechanics of the hydrogel solution. For instance, the effective amount of the catalyst agent may be at a concentration from least about 0.1-5 mM, 5-10 mM, 10-15 mM, 15-20 mM, 20-100 mM or greater than about 100 mM. The catalyst agent may be at any intervening concentration within a specific range. For instance, when the catalyst agent is from at least about 1-5 mM, the concentration of the catalyst agent may be about 1 mM, about 2 mM, about 3 mM, about 4 mM or about 5 mM. When the hydrogel solution is contacted with the competitor agent and the catalyst agent they may be in a specific ratio to one another. For instance, the competitor agent and the catalyst agent may be in a ratio of at least about 1:1 to at least about 1:100 or at least about 100:1 to at least about 1:1. The ratio of the competitor agent to the catalyst agent may be any intervening ratio with a specific range. For instance, if the competitor agent and the catalyst agent are in a ratio of at least about 1:1 to at least about 1:100 then the ratio may be at least about 1:1, 1:2, 1:3, 1:4, 1:5, 1:6, 1:7, 1:8, 1:9, 1:10, 1:20, 1:30, 1:40, 1:50, 1:60, 1:70, 1:80, 1:90 or at least about 1:100. if the competitor agent and the catalyst agent are in a ratio of at least about 100:1 to at least about 1:1 then the ratio may be at least about 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, or at least about 1:1.

In some embodiments, an effective amount of a competitor agent reduces the stiffness of the hydrogel solution by at least about 2 fold, 3 fold, 4 fold, 5 fold, 6 fold, 7 fold, 8 fold, 9 fold, 10 fold or greater than about 10 fold. In some embodiments, the effective amount of the competitor agent and the catalyst agent reduces the stiffness of the hydrogel solution by at least about 2 fold, 3 fold, 4 fold, 5 fold, 6 fold, 7 fold, 8 fold, 9 fold, 10 fold or greater than about 10 fold. The reduction in the stiffness of the hydrogel solution may last for a limited or an extended period of time. For instance, the reduction in the stiffness of the hydrogel gel solution may last for at least about 5 min, 10 min, 15 min. 20 min, 25 min, 30 min, 35 min, 40 min, 45 min, 50 min, 55 min, 60 min, 1.5 hours, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or greater than about 50 hours.

Also provided herein are methods for increasing the gelation time of a hydrogel solution. The methods comprise contacting the hydrogel solution with an effective amount of a competitor agent sufficient to alter the gelation time of the hydrogel solution. For instance, the effective amount of the competitor agent may be individually at a concentration from at least about 0.001-1 mM, 1-5 mM, 5-10 mM, 10-15 mM, 15-20 mM or greater than about 20 mM. The competitor agent may be at any intervening concentration within a specific range. For instance, when the competitor agent is from at least about 1-5 mM, the concentration of the competitor agent may be about 1 mM, about 2 mM, about 3 mM, about 4 mM or about 5 mM.

In some embodiments, the effective amount of a competitor agent increases the gelation time by at least about 10%, 20%, 30%, 40%, 50%, 60%, 70%, 80%, 90%, 100% or greater than about 100% increase in the gelation time relative to the absence of the competitor agent.

Also provided herein are methods for decreasing the gelation time of a hydrogel solution. The methods comprise contacting a hydrogel solution with an effective amount of a catalyst agent sufficient to decrease the gelation time of a hydrogel solution. The effective amount of a catalyst agent is any amount of a catalyst agent sufficient to decrease the gelation time of a hydrogel solution. For instance, the effective amount of the catalyst agent may be at a concentration from least about 0.1-1 mM, 1-5 mM, 5-10 mM, 10-15 mM, 15-20 mM, 20-25 mM, 25-100 mM or greater than about 100 mM. The catalyst agent may be at any intervening concentration within a specific range. For instance, when the catalyst agent is from at least about 1-5 mM, the concentration of the catalyst agent may be about 1 mM, about 2 mM, about 3 mM, about 4 mM or about 5 mM. In some embodiments, the effective amount of the catalyst agent decreases the gelation time by at least about 10%, 20%, 30%, 40%, 50%, 60%, 70%, 80%, 90%, 100% or greater than about 100% decrease in the gelation time relative to the absence of the catalyst agent.

In another aspect of the invention, a method is provided for the retrieval of cells or tissue from a 3D cell culture, the method comprising contacting the 3D cell culture with an effective amount of a competitor agent sufficient to dissociate the 3D cell culture and separating the cell or tissue from the 3D cell culture wherein the 3D cell culture comprises a hydrogel solution comprising a mixture of a first polymer and a second polymer wherein the first polymer is a modified with a functional group and the second polymer is modified with a functional group wherein the functional group of the first polymer can participate in reversible covalent bond formation with the functional group of the second polymer. In some embodiments, the first polymer is chemically modified hyaluronic acid (HA) and the second polymer is chemically modified elastin-like protein (ELP). In some embodiments, the method further comprises contacting the 3D cell culture with an effective amount of a catalyst agent sufficient to dissociate the 3D cell culture.

In another aspect of the invention, a method is provided for culturing cells or tissue in a 3D cell culture, the method comprising encapsulating an initiating population of mammalian cells in the hydrogel solution, contacting the 3D cell culture with an effective amount of a competitor agent sufficient to reduce the stiffness of the hydrogel solution, and maintaining the encapsulated cells in suitable medium.

The 3D cell culture of the present disclosure may comprise mammalian tissues or cells. In some embodiments, tissue, i.e. primary tissue, is obtained from a mammalian organ. The tissue may be from any mammalian species, e.g. human, equine, bovine, porcine, canine, feline, rodent, e.g. mice, rats, hamster, primate, etc. The mammal may be of any age, e.g. a fetus, neonate, juvenile, adult. The following are some non-limiting examples of tissues that may be obtained for the purposes of preparing organoids. Cells or tissue may be obtained by any convenient method, e.g. by biopsy, e.g. during endoscopy, during surgery, by needle, etc., or from cell lines, in vitro differentiation etc. If tissue, it is immersed in ice-cold buffered solution, e.g. PBS, Ham's F12, MEM, culture medium, etc. Pieces of tissue are minced to a size less than about 1 mm³, and may be dissociated to single cells. The minced tissue is mixed with a hydrogel of the disclosure. Subsequently, the cell-containing hydrogel is placed into suitable medium.

In some embodiments, tissue is grown in vitro from pluripotent stem cells, e.g. embryonic stem cells (ESCs), embryonic germ cells (EGCs), induced pluripotent stem cells (iPSCs). Any convenient method may be followed for the induction of the desired tissue from pluripotent stem cells. The engineered tissue can transferred to the hydrogel substrate.

The continued growth of explants may be confirmed by any convenient method, e.g. phase contrast microscopy, stereomicroscopy, histology, immunohistochemistry, electron microscopy, etc. In some instances, cellular ultrastructure and multi-lineage differentiation may be assessed. Ultrastructure of the intestinal explants in culture can be determined by performing Hematoxylin-eosin staining, PCNA staining, electron microscopy, and the like using methods known in the art. Multi-lineage differentiation can be determined by performing labeling with antibodies to terminal differentiation markers, e.g. as described in greater detail below. Antibodies to detect differentiation markers are commercially available from a number of sources.

In some embodiments, the cells in the cultured explants may be experimentally modified. For example, the explant cells may be modified by exposure to viral or bacterial pathogens, e.g. to develop a reagent for experiments to assess the anti-viral or anti-bacterial effects of therapeutic agents. The explant cells may be modified by altering patterns of gene expression, e.g. by providing reprogramming factors to induce pluripotency or otherwise alter differentiation potential, or to determine the effect of a gain or loss of gene activity on the ability of cells to form an explant culture or on the ability of cells to undergo tumor transformation. The explant cells may be modified such that they are transformed into proto-oncogenic or oncogenic cells, e.g. by providing cancer drivers—oncogenic factors or inhibitors of tumor suppressor genes, e.g. nucleic acids for the overexpression of Kras.sup.G12D; nucleic acids that suppress expression of APC, p53, or Smad4, etc., for example, to assess the effects of therapeutic agents on tumors.

Organoids prepared by the subject methods find use in many applications. For example, cancer, ischemia, congenital syndromes, trauma, and inflammation can produce functional loss or mandate physical resection of large sections of patient tissue extensive enough to compromise organ physiology. The ability to grow explants of mammalian tissue in vitro to be placed back into such patients or to be used as a source of tissue-specific stem cells for transplantation into such patients is a valuable treatment option. The use of competitor and catalyst agents as disclosed herein also for the efficient extraction of organoids from 3D cell cultures. Such cells can augment the ex vivo expansion of tissue, providing an autologous source of engineered tissue and/or tissue stem cells. As another example, organoids prepared by the subject methods may be used to predict the responsiveness of an individual, e.g. an individual with cancer, with an infection, etc., to a therapy. As another example, organoids prepared by the subject methods may be used in basic research, e.g. to better understand the basis of disease and in drug discovery, e.g. as reagents in screens such as those described further below. Organoids are also useful for assessing the pharmacokinetics and pharmacodynamics of an agent, e.g. the ability of a mammalian tissue to absorb an active agent, the cytotoxicity of agents on primary mammalian tissue or on oncogenic mammalian tissue, etc.

The organoids of the present disclosure may be any organoids known in the art including, without limitation, neural organoids, hepatic organoids, intestinal organoids, gastric organoids, lung organoids, vascular organoids, retina organoids, cardiac organoids, kidney organoids, etc. Methods of generating the organoids disclosed above are known in the art and have been described in, for example, Zhu et al. (Stem Cell Rev Rep. 2022 December;18 (8):2593-2605), Romero-Guevara et al. (Front Physiol. 2020; 11:563981), Wahlin et al. (Sci Rep. 2017 Apr. 10; 7 (1):766), van den Berg et al. (Stem Cell Reports. 2018 Mar. 13;10(3):751-765), Hu et al. (Cell. 2018 Nov. 29;175(6):1591-1606.e19), Hild et al. (Curr Protoc Stem Cell Biol. 2016 May 12;37:IE.9.1-IE.9.15), Salahudeen et al. (Nature. 2020 December;588 (7839):670-675), Barkauskas et al. (J Clin Invest. 2013 July;123(7):3025-36), McCracken (Nature. 2014 Dec. 18;516 (7531):400-4), Spence et al. (Nature. 2011 Feb. 3;470 (7332):105-9), Hofbauer et al. (Cell. 2021 Jun. 10;184(12):3299-3317.e22), Wimmer et al. (Nature. 2019 January;565 (7740):505-510), and Revah et al. Nature. 2022 October;610 (7931):319-326, each specifically incorporated by reference herein.

The hydrogel solutions disclosed herein provide particular benefits to the cells cultured therein. In some embodiments, the competitor agent is not cytotoxic and does not impair cell, tissue, or organoid growth. In some embodiments, the competitor agent does not impair or alter the morphology of the cell, tissue, or organoid relative to the cell, tissue, or organoid cultured in the hydrogel solution without the competitor agent. In some embodiments, the presence of the competitor agent in the hydrogel solution increases the efficiency of the organoid formation relative to a hydrogel solution that does not contain the competitor agent.

### Compositions

Also, compositions of the present disclosure are provided. In general, subject compositions contain the components, e.g., as described above. For instance, the composition may contain the hydrogel solution and the competitor agent disclosed herein. In some embodiments, the composition comprises the catalyst agent. In some embodiments, the composition further comprises cells, tissues or organoids as described above. In some embodiments, the composition further comprises a culture medium sufficient to support the growth of cells, tissue or organoids that may be contained therein.

The hydrogel solution comprises a mixture of a first polymer and a second polymer wherein the first polymer is modified with a first functional group and the second polymer is modified with a second functional group wherein the first functional group of the first polymer can participate in reversible covalent bond formation with the second functional group of the second polymer. In some embodiments, the first polymer is chemically modified hyaluronic acid (HA) and the second polymer is chemically modified elastin-like protein (ELP). In some embodiments, the first polymer is chemically modified with the first functional group and the second polymer is chemically modified with the second functional group. In some embodiments, the first functional group comprises an aldehyde or benzaldehyde-containing functional group and the second functional group comprises a hydrazine-containing functional group. In some embodiments, the first functional group comprises a hydrazine-containing functional group and the second functional group comprises an aldehyde or benzaldehyde-containing functional group.

The competitor agent may be any agent that comprises a functional group that is analogous to the first or second functional group of the first or the second polymer. In some embodiments, the competitor agent comprises an aldehyde, a benzaldehyde or a hydrazine functional group. In some embodiments, the competitor agent is selected from the group of 2-hydrazinoethanol, benzaldehyde, 4-hydrazinobenzoic acid, hydrazinoacetic acid, butyraldehyde, and derivatives thereof.

The catalyst agent may be any agent that increases the forward and reverse reaction rate of the reversible covalent bond formation in the hydrogel solution. In some embodiments, the catalyst agent is selected from the group of 2-(aminomethyl) imidazoles, N,N-dimethylethylenediamines, 2-aminobenzenephosphonic acids, 2-aminophenols and derivatives thereof.

### Kits

Also, kits for practicing the methods described in the present disclosure. In general, subject kits may contain the components, e.g., as described above. For instance, the kit may contain the hydrogel solution and the competitor agent disclosed herein. In some embodiments, the kit comprises the catalyst agent. In some embodiments, the kit comprises culture medium to facilitate the culturing of a specific cell, tissue, or organoid type.

A subject kit can include any combination of components for performing the methods of the present disclosure. The components of a subject kit can be present as a mixture or can be separate entities. In some cases, components are present as a lyophilized mixture. In some cases, the components are present as a liquid mixture. Components of a subject kit can be in the same or separate containers, in any combination.

The subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer-readable medium, e.g., diskette, compact disk (CD), flash drive, and the like, on which the information has been recorded. Yet another form of these instructions that may be present is a website address which may be used via the internet to access the information at a remote site.

### EXAMPLES

### Example 1

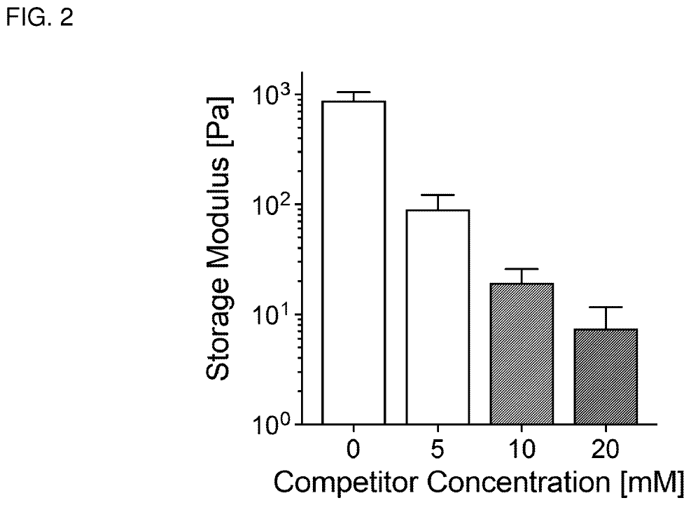

As shown in FIG. 2, the competitor agent, hydrazinoacetic acid, was added to HELP hydrogels in four separate final concentrations, 0 mM, 5 mM, 10 mM and 20 mM. The storage modulus of the HELP hydrogels were measured using oscillatory shear rheology. Hydrazinoacetic acid reduced the storage modulus in a dose dependent manner.

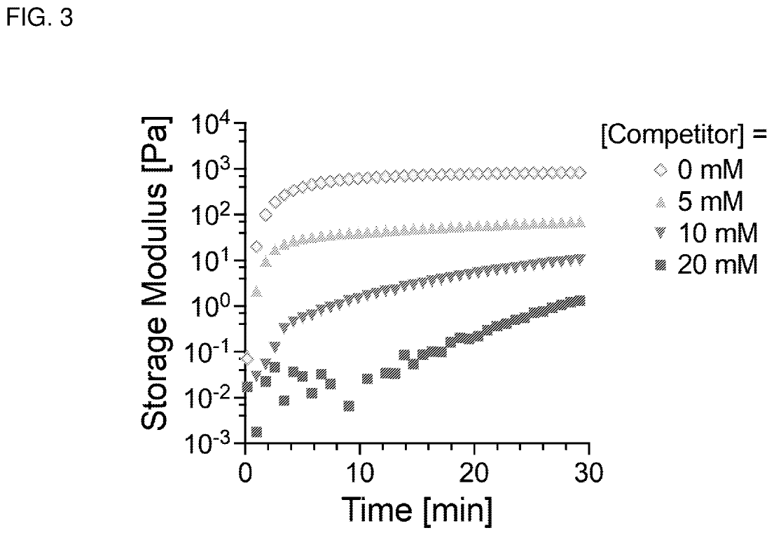

FIG. 3 shows the change in the storage modulus of HELP hydrogels over time. The HELP hydrogel composition was 1 wt % ELP-Hydrazine (fully modified) and 1 wt % HA-Aldehyde (12% modified). Hydrazinoacetic acid was added to HELP hydrogels in four separate final concentrations, 0 mM, 5 mM, 10 mM and 20 mM and the storage modulus was measured over the course of 30 minutes. Hydrazinoacetic acid both reduced the storage modulus of HELP hydrogels in a dose dependent manner and increased the time to reach the plateau storage modulus, where the higher the concentration of hydrazinoacetic acid the slower the increase in storage modulus over time.

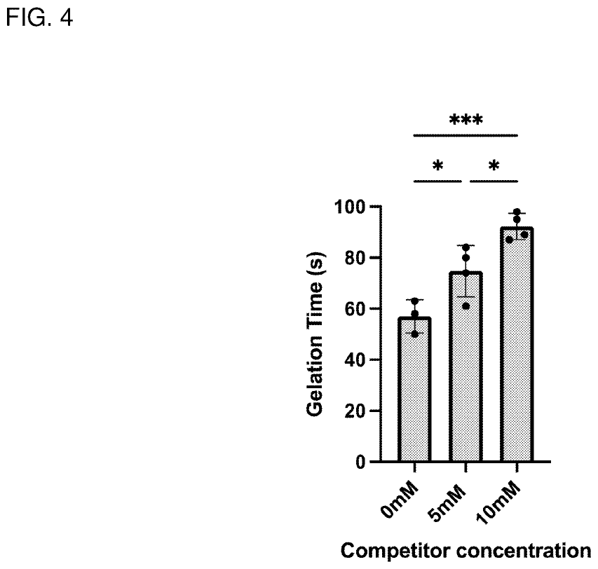

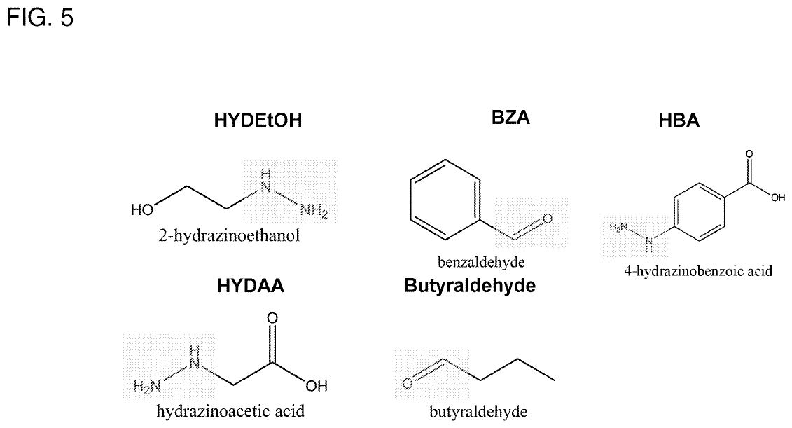

As shown in FIG. 4, hydrazinoacetic acid was added to HELP hydrogels in three separate final concentrations, 0 mM, 5 mM, and 10 mM and the gelation time of the HELP hydrogel was measured. The HELP hydrogel composition was 2 wt % ELP-Hydrazine (fully modified) and 1 wt % HA-Benzaldehyde (30% modified). Hydrazinoacetic acid increased the gelation time in a dose dependent manner where 10 mM hydrazinoacetic acid increased the gelation time by approximately 30 seconds.

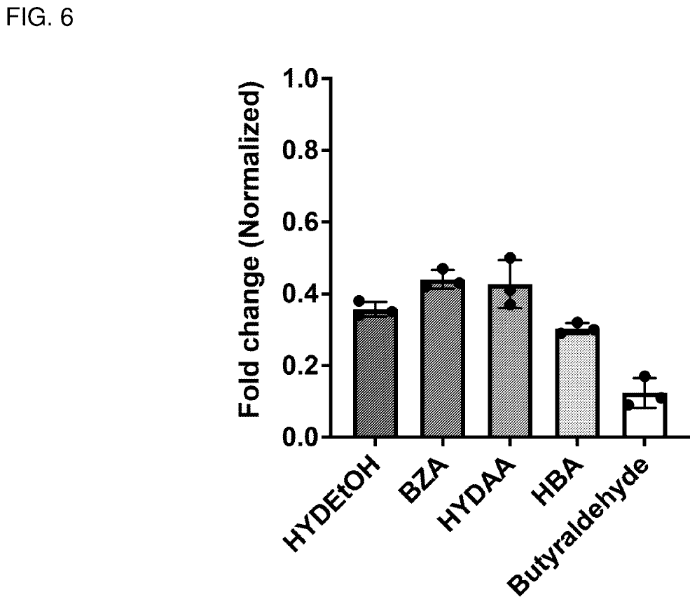

FIG. 6 shows the comparison of a range of competitor agents on the fold change in HELP hydrogel storage modulus relative to no competitor agent. The HELP hydrogel composition was 2 wt % ELP-Hydrazine (fully modified) and 1 wt % HA-Benzaldehyde (30% modified). The competitor agents used were 2-hydrazinoethanol (HYDEtOH), benzaldehyde (BZA), hydrazinoacetic acid (HYDAA), 4-hydrazinobenzoic acid (HBA), and butyraldehyde. Each competitor agent was added to HELP hydrogels at a final concentration of 10 mM.

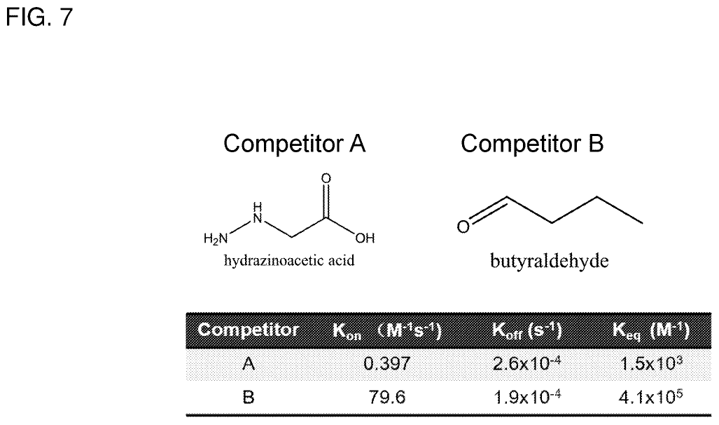

As shown in FIG. 7, the forward and reverse rate constants and the equilibrium constant of two separate competitor agents to the functional groups of the HELP system. The competitor agents used were HYDAA and butyraldehyde.

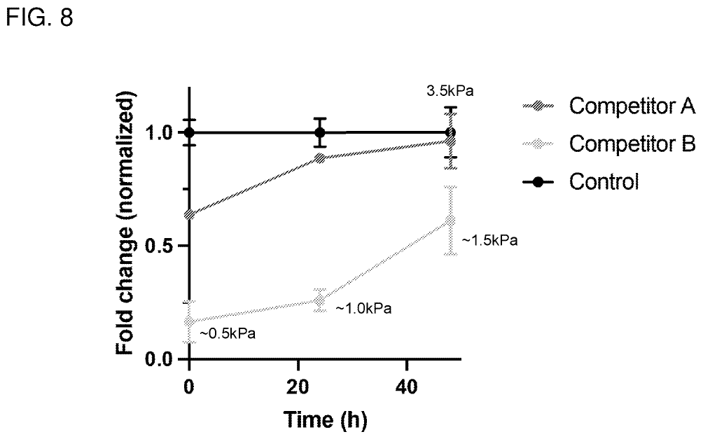

FIG. 8 shows the fold change in the storage modulus of the HELP hydrogels in response to HYDAA and butyraldehyde over time. The composition of the HELP hydrogel was 2 wt % ELP-Hydrazine (fully modified) and 1 wt % HA-Benzaldehyde (30% modified). HYDAA (competitor A) and butyraldehyde (competitor B) were added to HELP hydrogels and the storage modulus of HELP hydrogels was measured and the fold change in storage modulus was calculated overtime relative to HELP hydrogels without a competitor agent.

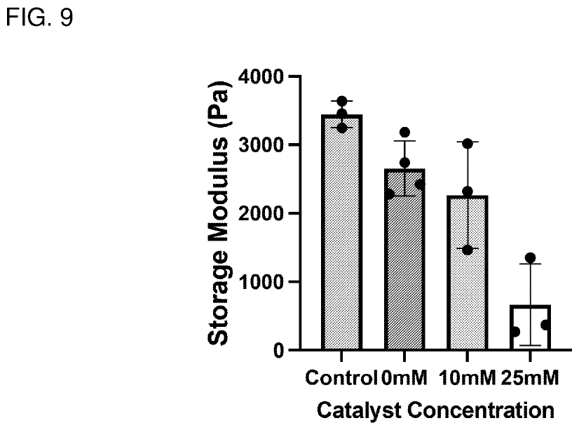

As shown in FIG. 9, the storage modulus of HELP hydrogels was measured in response to varying concentrations of catalyst and competitor agent after 24 hours of allowing the competitor and catalyst to diffuse into the preformed hydrogel. The catalyst agent was 2-(Aminomethyl)benzimidazole and the competitor agent was butyraldehyde. The competitor agent was added at HELP hydrogels at a final concentration of 10 mM and the catalyst agent was added to a final concentration of 0.10 and 25 mM. The composition of the HELP hydrogel was 2 wt % ELP-Hydrazine (fully modified) and 1 wt % HA-Benzaldehyde (30% modified). The storage modulus was reduced in a dose-dependent manner in response to increasing concentrations of catalyst with a constant competitor agent concentration.

### Example 2

### Introduction

Hydrated, polymeric scaffolds, termed hydrogels, are widely used for tissue engineering and regenerative medicine approaches. (Lee and Mooney 2001) Hydrogels are especially advantageous for three-dimensional cell culture as they are largely composed of water (˜90%) and are readily tuned to mimic essential features of the native tissue microenvironment. (Peppas, Hilt et al. 2006) Biomechanical and biochemical cues, such as stiffness and cell-adhesive ligand density, can be intentionally designed into the hydrogel by controlling initial synthesis parameters. (Drury and Mooney 2003, Burdick and Stevens 2005, Wang, Cai et al. 2014, Ho, Keown et al. 2017) Another important property that can be incorporated into hydrogel design is the ability of the material to be remodeled by encapsulated cells, which reflects the way that cells remodel the native microenvironment. (Wang and Heilshorn 2015, Gilchrist and Harley 2021, Rizwan, Baker et al. 2021, Saraswathibhatla, Indana et al. 2023) Cell-mediated hydrogel remodeling is implicated in driving cell phenotype, including cell migration and stem cell differentiation. (Chaudhuri, Gu et al. 2015, Chaudhuri, Gu et al. 2016, Lou, Stowers et al. 2018, Gilchrist, Lee et al. 2019) In chemical hydrogels with static crosslinks, remodelability is often achieved by incorporating hydrolytically cleavable components into the material; however, this process is irreversible. (Raeber, Lutolf et al. 2005, Stevens, Miller et al. 2015, Muir and Burdick 2021) In contrast, physical hydrogels with dynamic crosslinks formed through physical interactions between polymer chains are inherently remodelable, but the resulting materials are typically quite weak (<0.1 kPa). (Wong Po Foo, Lee et al. 2009, Hu, Wang et al. 2019, Steele, Stapleton et al. 2019, Muir and Burdick 2021) As an alternative approach, dynamic covalent chemistry (DCC) has emerged as a crosslinking strategy that enables synthesis of stiff (>1 kPa) (Mckinnon, Domaille et al. 2014, Madl and Heilshorn 2019) and remodelable hydrogels for tissue engineering applications. (Rizwan, Baker et al. 2021) While covalent bonds are generally thought of as static, DCC bonds are strong and reversible, making them an ideal choice for synthesis of remodelable hydrogels. (Rosales and Anseth 2016, Tang, Richardson et al. 2021)

Hydrazone chemistry is a particularly useful class of DCC for tissue engineering approaches. Compared to other DCC strategies, the forward and reverse reactions rates of hydrazone chemistry are appreciable at physiological conditions (˜10⁻¹-10¹ s⁻¹). (Kool, Park et al. 2013, Saito, Noda et al. 2015) Additionally, the crosslinking reaction is spontaneous, (Rowan, Cantrill et al. 2002) requiring no ultraviolet light or radical initiation, which eliminates confounding reactive radicals that can occur in other crosslinking strategies. (Gilchrist, Serrano et al. 2021) Despite these advantages, hydrazone-crosslinked hydrogels have some limitations. Namely, the reaction proceeds rapidly, which quickly produces a percolated network of polymers that form a gel. This rapid gelation rate can result in inhomogeneous mixing of the polymer precursors and heterogeneous presentation of biochemical and biomechanical properties to encapsulated cells. Additionally, the reversible nature of hydrazone crosslinks can lead to erosion of the hydrogel over time, as uncrosslinked polymers diffuse into the surrounding solution, resulting in loss of mass and mechanical stability. (Kalia and Raines 2008, Wang, Highley et al. 2018, Tang, Richardson et al. 2021) To overcome this issue, DCC has been coupled with self-assembling proteins, such as collagen or elastin, that form supramolecular networks. (Wang, Zhu et al. 2017, Lou, Stowers et al. 2018) The structures formed by these self-assembling proteins create a network of secondary crosslinks that can minimize erosion and improve hydrogel stability.

Specifically, recombinant elastin-like protein (ELP) has been used in conjunction with DCC to form stable hydrogels. ELP displays an inverse phase transition and forms protein-rich aggregates above a lower critical solution temperature. (Navarro, Huang et al. 2022) This protein aggregation creates a secondary network that stabilizes the DCC hydrogel for extended cell culture. (Wang, Zhu et al. 2017) ELPs are highly reproducible and enable intrinsic presentation of biochemical and biomechanical cues through defined sequences of amino acids. (Madl, Katz et al. 2016, LeSavage, Suhar et al. 2018) To create a biomimetic hydrogel, ELP can be combined with recombinant hyaluronic acid (HA), a linear polysaccharide that is broadly expressed in many native tissues. (Burdick and Prestwich 2011, Dicker, Gurski et al. 2014) Using hydrazone chemistry, hydrogels made of HA and ELP (termed HELP) are stable and have highly tunable biochemical and biomechanical properties. (Wang, Zhu et al. 2017, Zhu, Wang et al. 2017, Suhar, Doulames et al. 2022) These HELP materials have been used to successfully culture patient-derived organoids without the need for animal-derived matrices. (Hunt, Klett et al. 2021, LeSavage, Gilchrist et al. 2022)

Previous work has reported the injectability of DCC-crosslinked hydrogels, including HELP (Hull, Lou et al. 2023) and polyethylene glycol (PEG)-based systems (Lou and Xia 2022), for bioink development and cell delivery applications, respectively. In both material systems, the extrusion of the hydrogel was aided by incorporation of a small molecule competitor (hydrazinoacetic acid). The competitor transiently disrupts DCC crosslinks, reducing the crosslinking density and increasing extrudability. Post-extrusion, the competitor can freely diffuse out of the hydrogel, resulting in a higher crosslinking density and stiffer hydrogel. These proof-of-concept studies demonstrated the utility of a biocompatible competitor to enable extrusion of cell-laden, DCC hydrogels. Given the promise of competitors to transiently alter the mechanical properties of DCC hydrogels, we conjectured that development of a library of competitors would allow us to access a wide range of material properties. Specifically, we hypothesized that competitors with a range of molecular-level properties (e.g. reaction kinetics and thermodynamic constants) would allow us to reproducibly and predictably tune macro-scale hydrogel properties, (e.g. the gelation time, sol-gel behavior, and gel stiffness).

Towards that goal, here we identify a library of competitors that display water solubility at physiological conditions and have distinct chemical structures that impact their hydrazone reaction kinetics and thermodynamics. Using models grounded in polymer physics, we relate the molecular-level parameters of the competitors to their effects on the macromolecular-network properties to predict the sol-gel phase behavior of HELP hydrogels. We hypothesize that by selecting the appropriate competitor concentration and chemical structure, we can fine-tune the crosslinking density and achieve hydrogels with a wide range of mechanical properties while maintaining a constant polymer concentration. Additionally, to address the rapid gelation of hydrazone-based hydrogels, we take advantage of the transient disruption of crosslinks to predictably increase the gelation time, resulting in improved homogeneity of hydrogels. Finally, we demonstrate the usefulness and cell-compatibility of this system by encapsulating patient-derived intestinal stem cells in homogenous HELP hydrogels to successfully grow human intestinal organoids.

### Results and Discussion

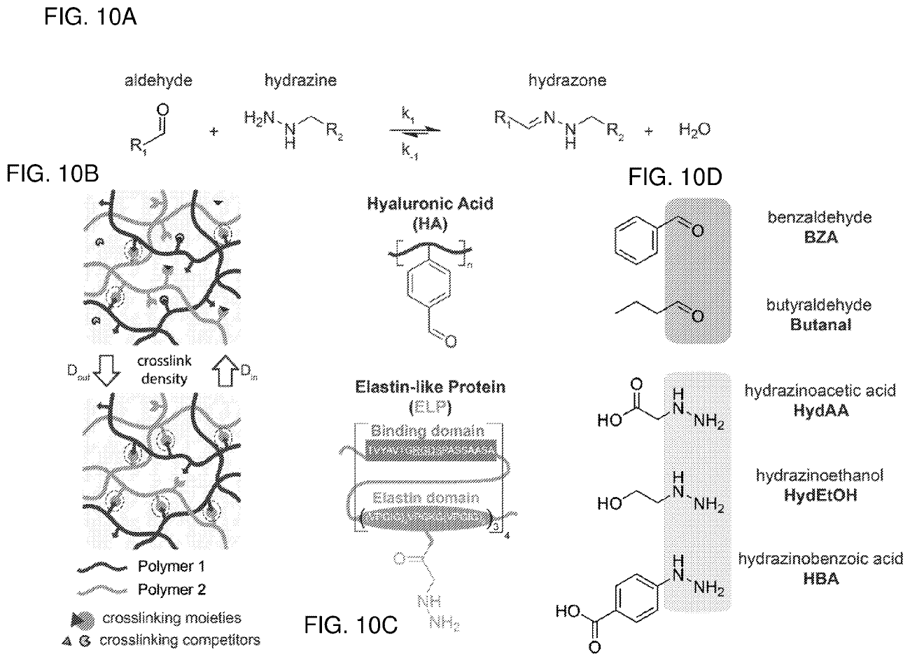

The formation of a hydrazone bond from aldehyde and hydrazine is facile, reversible, and widely used for crosslinking of dynamic hydrogels (FIG. 10A). (Teng, Chen et al. 2019) The reversibility of the reaction is characterized by the forward (k₁) and reverse (k₋₁) reaction rate constants. The speed of these reactions is governed by the putative rate limiting step of an intermediate aldehyde-hydrazine tetrahedral molecule, which can be stabilized or destabilized by the corresponding side groups (R₁, R₂). (FIG. 10A) (Jencks 2007, Kolmel and Kool 2017) The stability of the hydrazone bond is characterized by the thermodynamic equilibrium constant, K_{eq}, which is the ratio between k₁/k−1. As such, K_{eq} can differ over several orders of magnitude, depending on the chemistry of the side groups. (Kool, Park et al. 2013)

Due to the reversibility of DCC crosslinks, at any given time there will be a proportion of aldehydes and hydrazines that remain available and not involved in hydrazone bond formation. We can take advantage of these unreacted crosslinking sites by utilizing diffusible, small molecules that can react with either aldehyde or hydrazine and prevent crosslink formation. By competing for crosslinking sites, the small molecules, termed competitors, reduce the overall crosslink density (ρₓ) and hydrogel modulus (FIG. 10B). Competitors that reversibly react can be used to transiently disrupt the crosslinking density and then diffuse out of the hydrogel, leading to recovery of the maximum crosslinking density. (Lou and Xia 2022, Hull, Lou et al. 2023)

Previously, we have used a hydrazine-based competitor in a recombinant DCC-crosslinked HELP hydrogel, (Hull, Lou et al. 2023) Here, HA and ELP are modified along their backbone with a benzaldehyde (HA-BZA) or hydrazine (ELP-HYD), respectively (FIG. 10C). Combining the chemically modified HA and ELP results in a hydrazone crosslinked HELP hydrogel that is both biologically active and stable for extended culture. (Wang, Zhu et al. 2017, Hunt, Klett et al. 2021, LeSavage, Gilchrist et al. 2022, Shayan, Huang et al. 2023) Using this system, we demonstrated use of hydrazinoacetic acid as a competitor to transiently disrupt crosslinking density for the bioprinting of HELP. (Hull, Lou et al. 2023)

Inspired by the use of an alkyl hydrazine competitor to alter hydrogel network properties, we recognized the potential to utilize the side group of the competitor to modulate the reaction kinetics and fine-tune the equilibrium crosslinking density. We hypothesized that by altering the competitor side groups, we could access a broader range of macromolecular network properties and predictably fine-tune hydrogel mechanics. To this end, we identified a library of small molecule competitors that are sufficiently soluble in aqueous solution and contain either aldehyde or hydrazine functionality to react with ELP-HYD or HA-BZA, respectively (FIG. 10D). Within each aldehyde or hydrazine functional group, we selected competitors that contain either an alkyl or aryl (i.e. aromatic) side group to encompass a broad spectrum of reaction kinetics and thermodynamic equilibria to achieve precise control over hydrogel properties.

To quantify the extent that side groups impact reaction parameters, the forward (k₁) and reverse (k₋₁) reaction rates were measured for each competitor at 50 μM (Table 2). For these reactions, the complementary reactant was chosen to be 50 μM of either benzaldehyde (BZA) or hydrazinoacetic acid (HydAA), because they are the crosslinking functionalities of HELP, and can provide key insight into DCC interactions without the complexity of the full HA and ELP polymers. (Winne, Leibler et al. 2019) The formation of a hydrazone bond was observed via absorbance with UV spectroscopy and converted to a concentration over time. An analytical solution to a second order reaction rate, assuming equal concentrations of reactants, was fit to the concentration as a function of time. (Dirksen, Dirksen et al. 2006, Mckinnon, Domaille et al. 2014)

TABLE 2
Reaction rates of each competitor with either benzaldehyde or hydrazinoacetic acid.

| Functionality | k₁ | k₋₁ (×10⁻⁴) | K_{eq} |
|---|---|---|---|
| | M⁻¹ s⁻¹ | s⁻¹ | M⁻¹ |
| Aldehyde | | | |
| Benzaldehyde* (BZA) | 0.276 (0.070) | 1.71 (0.280) | 1.61 × 10³ (0.484 × 10³) |
| Butyraldehyde (Butanal) | 59.9 (5.49) | 4.49 (1.07) | 1.33 × 10⁵ (0.34 × 10⁵) |
| Hydrazine | | | |
| Hydrazinobenzoic acid (HBA) | 2.10 (0.40) | 1.00 (0.077) | 2.09 × 10⁴ (0.43 × 10⁴) |
| Hydrazinoethanol (HydEtOH) | 0.690 (0.301) | 1.12 (0.469) | 6.18 × 10³ (3.74 × 10³) |
| Hydrazinoacetic acid* (HydAA) | 0.276 (0.070) | 1.71 (0.280) | 1.61 × 10³ (0.484 × 10³) |

Forward (k₁) and reverse (k₋₁) reaction rates listed as mean (standard deviation) of n = 4 replicates. Reactions were performed in saline solution with equal concentrations (50 μM) of competitors and model reactants (BZA and HydAA) at 25° C.
*indicates repeat values of BZA and HydAA.

As expected, the competitor's side group has a significant impact on reaction rates (k₁, k₋₁) and product formation (K_{eq}). Comparing the reaction rates of the aldehyde competitors, alkyl butyraldehyde (Butanal) has faster reaction rates (k₁=59.9±5.49, k₋₁=4.49±1.07×10⁻⁴) and a more favored hydrazone production (K_{eq}˜10⁵) compared to the aromatic aldehyde (BZA) (k₁=0.276±0.070, k₋₁=1.71±0.280×10⁻⁴, K_{eq}˜10³). In fact, the single aromatic hydrazone reactions of BZA, hydrazinoethanol (HydEtOH), and HydAA, have similar forward and reverse reaction rates and equilibrium constants (k₁˜10⁻¹, k⁻¹˜10⁻⁴, K_{eq}˜10³). A double aromatic hydrazone, such as the reaction between BZA and hydrazinobenzoic acid (HBA), resulted in rates that were intermediate between an alkyl and single aromatic hydrazone (k₁=2.10±0.400, k₋₁=1.00±0.077×10⁻⁴, K_{eq}˜10⁴). These results are consistent with literature, where an alkyl hydrazone (HydAA+Butanal) has the fastest reaction rates and was the most favored reaction compared to the aromatic hydrazone (HydAA+BZA). (Kool, Park et al. 2013) Altering the side groups of the competitors, the equilibrium constant can be tuned over two orders of magnitude.

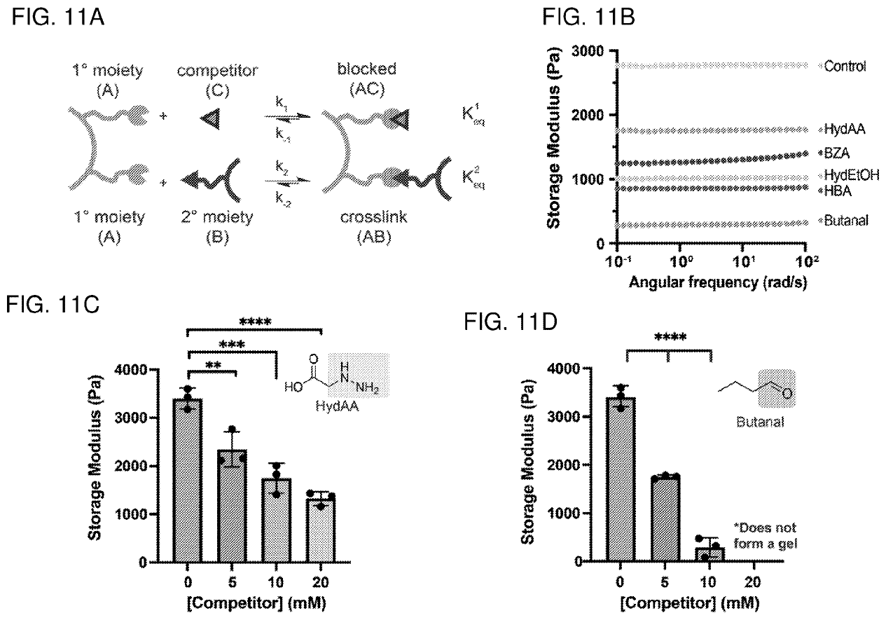

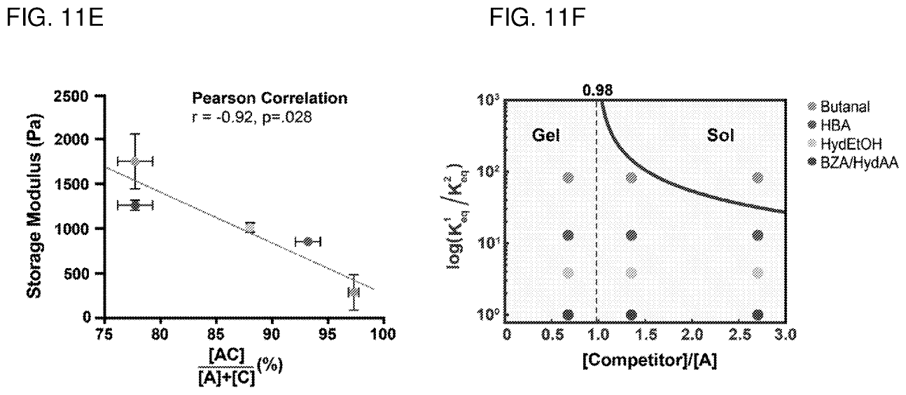

To initially assess the impact of a competitor on hydrogel stiffness, it was proposed that the effect on crosslinking density will depend upon the relative strength of the two competing reactions: (1) crosslinking-site and competitor and (2) crosslinking reaction (FIG. 11A). It was predict that as the relative strength of the competitor reaction (K_{eq}¹) to crosslinking reaction (K_{eq}²) increases, there will be a greater disruption of crosslinks. To validate this, the impact of competitor K_{eq} on the modulus of HELP formed from precursor solutions of HA-BZA and ELP-HYD at a 1:1 BZA to HYD ratio was determined. To minimize off-target reactions, competitors were added to the precursors solution that has the same functionality, i.e., hydrazine competitors added to ELP-HYD, aldehyde competitors added to HA-BZA. At 10 mM of competitor, which represents a ratio of 4:3 of competitor to crosslinking sites, all competitors led to a significant decrease in the HELP modulus compared to the 0 mM control (FIG. 11B). Based upon the hypothesis that the relative strength of the reactions will dictate crosslinking disruption, a larger decrease in modulus with competitors that result in an aromatic hydrazone (K_{eq}¹/K_{eq}²˜10⁰), double aromatic hydrazone (K_{eq}¹/K_{eq}²˜10¹) and alkyl hydrazone (K_{eq}¹/K_{eq}²˜10²) should be seen. Consistent with the prediction, the least favored reaction HydAA (K_{eq}¹/K_{eq}²˜10⁰), had the least impact on modulus (1.75±0.3 kPa); whereas, Butanal, which has the largest equilibrium constant (K_{eq}¹/K_{eq}²˜10²) resulted in the largest drop in modulus (0.29±0.2 kPa). HBA, a double aromatic hydrazone, had a decrease in modulus in-between Butanal and HydAA (0.86±0.04 kPa).

The hydrogel modulus response to increasing concentrations of competitors was next examined. The decrease in modulus is dependent upon the concentration of the competitor. To examine this dose-dependent response, the competitors with the lowest (HydAA, aromatic hydrazone) and highest (Butanal, alkyl hydrazone) K_{eq} were selected. Increasing the concentration of HydAA from 5, 10, and 20 mM further reduced the crosslinking density and modulus (2.3, 1.8, 1.3 kPa) in a concentration dependent manner (FIG. 11C). A similar response with Butanal was seen as we increase the concentration from 5 to 10 mM (FIG. 11D). Further increasing the concentration of Butanal to 20 mM can completely overwhelm the crosslinks and disrupt formation of a hydrogel.

As a first approximation of the impact of the competitor K_{eq} on hydrogel modulus, it was predicted that the percentage of competitors that would be bound to the biopolymer at equilibrium. Using the reaction kinetic parameters, the equilibrium ratio of hydrazone present in the competitor and the model molecules (BZA and HydAA) reaction was calculated. Comparing this proportion to the hydrogel modulus, it was seen that the extent of modulus decrease is strongly correlated (r=−0.092) with the reaction kinetics of the competitor (FIG. 11E). This demonstrates that as the competitor hydrazone bond becomes more favored (higher K_{eq}), the modulus of the hydrogel decreases more. Notably, the prediction of hydrazone proportion uses only the competitor reaction and does not take into account the crosslinking reaction. This analysis illustrates that while the modulus cannot be precisely, (Winne, Leibler et al. 2019) insightful conclusions can be drawn about macromolecular properties using simple chemical parameters. Using reaction kinetics, it can be predicted that in the HELP system, an alkyl aldehyde competitor, such as Butanal, will lead to a significantly greater decrease in modulus, compared to alkyl hydrazine competitor, such as HydAA, leading to a 0.1 and 0.6 fold change in modulus, respectively.

In addition to predicting stiffness, it was desired to predict the limits of competitor reactivity (K_{eq}) and concentration that would still allow for hydrogel formation. A gel is formed when sufficient crosslinking sites have reacted to form a percolating network (infinite polymer). (De Gennes 1976, Rubinstein and Colby 2003) In mean-field theory, the gelation point occurs when the crosslinking reaction has proceeded to a critical extent to form a percolated network. Reactions that have not reached the critical extent of reaction (p_{c}) are in the sol phase, while those that reach p_{c} are in a gel phase. It was hypothesized that there should exist some competitor system that does not allow the HELP crosslinking reaction (FIG. 11A) to reach the critical extent of reaction. Using percolation theory, a phase diagram was assembled to delineate combinations of relative reaction strength and competitor concentration that produce a gel or sol. By combining the two competing reactions of competitor and crosslinking (FIG. 11A), the relative strength of the reactions (K_{eq}¹/K_{eq}²) and initial concentration of the reactants ([A], [B], [C]) can be related to the extent of crosslinking reaction (p; see below for full derivation):

$$( 1 ) \to \frac{{K}_{eq \text{.1}}}{{K}_{eq \text{.2}}} = \frac{( {[ A ]}_{0} - \frac{p}{{K}_{2} ( 1 - p )} - {p [ B ]}_{0} ) ( 1 - p )}{( {[ C ]}_{0} - ( {[ A ]}_{0} - \frac{p}{{K}_{2} ( 1 - p )} - {p [ B ]}_{0} ) ) p}$$

To determine the required critical extent of reaction, we again employ mean-field theory to express p_{c} in terms of the number of potential crosslinking sites on the polymer,

$${p}_{c} = \frac{1}{f - 1} ,$$

i.e., p_{c} is inversely related to the functionality of the polymer, f. (Rubinstein and Colby 2003) The high functionality of the HELP system, taken as the average functionality of HA-BZA and ELP-HYD, leads to a small critical extent of reaction. The implication of this is that regardless of how favored the competitor reaction is, so long as the competitor concentration is 98% of the crosslinking sites, there will be sufficient functionality to permit a percolated network at equilibrium (see below). Combining equation (1) with the definition of the critical extent of reaction, we assembled a phase diagram that identifies competitor conditions that prevent the crosslinking reaction from forming a gel within the HELP system. Inputting the reaction parameters and concentrations of each of the competitors, we predicted the formation of a gel or sol for the library of competitors. For a given competitor, with sufficiently high K_{eq} (Butanal), a concentration ˜1.7× the concentration of crosslinking sites results in a system that will not gel. This predicted behavior is validated by the inhibition of HELP gel formation with the addition of Butanal at 20 mM (2.7×). By combining reaction kinetics with mean-field theory of gelation, the concentration of competitors that will prevent gelation from occurring can be predicted. Thus, the phase space of possible hydrogel moduli are set by the range of possible competitor concentrations and K_{eq}. The maximum gel stiffness is achieved when no competitor is present. The minimum gel stiffness possible is achieved when the competitor is just below the critical concentration that prevents formation of a percolated network (p_{c}). Between these two values, the entire phase space of possible moduli can be accessed in a continuous manner by simply changing the competitor concentration.

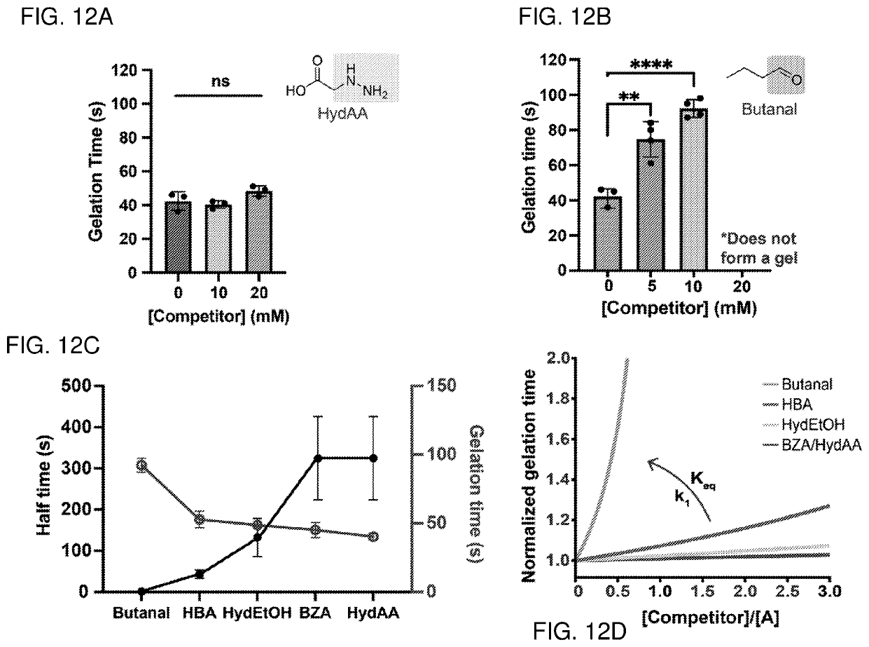

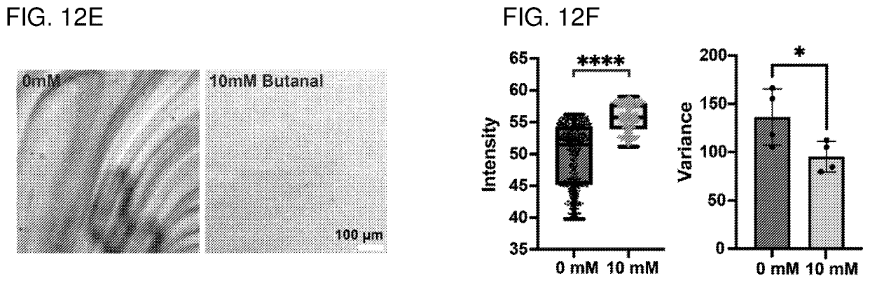

The rapid gelation of DCC hydrogels was addressed, which resulted in heterogeneous presentation of biochemical and biomechanical cues and is a key limitation of hydrazone-crosslinked hydrogels. Based upon the observed and predicted effect of the competitor on hydrogel modulus, it was reasoned that transient disruption of crosslinking would alter gelation kinetics. The competitor kinetics delays the time to reach a percolated polymer network in a dose-dependent manner. Addition of a high concentration (20 mM) of a relatively slow reacting competitor K_{eq} competitor (HydAA, k₁˜10⁻¹, K_{eq}˜ 10³) results in a modest and nonsignificant increase in gelation time (t_{g}=48.3±3.1 s), though it softened the hydrogel modulus from ˜3 kPa to 1.3 kPa. (FIG. 12A). Comparatively, a rapidly reacting competitor (Butunal, k₁˜ 10¹, K_{eq}˜ 10⁵) significantly increased the gelation time from 42.3±5.5 to 74.8±10.0 and 92.3±5.1 s with increasing concentration (0, 5, 10 mM; FIG. 12B). The difference in gelation times can be attributed to the difference in forward reaction rate and equilibrium constants of the competitors, which both differ by over 2 orders of magnitude between Butanal and HydAA. This is advantageous as 5 mM Butanal and 10 mM HydAA both led to a decrease in modulus from ˜3 kPa to 1.75 kPa; however, 5 mM Butanal increased the gelation time by ˜1.7 fold while HydAA did not significantly impact gelation. This demonstrates that by tuning competitor kinetics and concentration, two hydrogel material properties (modulus and gelation time) can be tuned

Using the kinetic reaction parameters, the theoretical half-time (t_{1/2}) to was calculated to reach equilibrium of the competitor reaction at a concentration of 10 mM (FIG. 12C). The BZA and HydAA reactions are predicted to reach equilibrium at the longest time (t_{1/2}=254±80 s), compared to the relatively fast Butanal reaction (t_{1/2}=1.59±0.14 s). This is consistent with the rationale that Butanal increases the gelation time because it is capable of rapidly reacting with the crosslinking sites, preventing them from participating in gelation. This is further validated by hydrogel gelation times using each of the competitors, where faster reactions (smaller t_{1/2}) resulted in slower gelation times (larger t_{g}). Using a simplified half-time model of just the competitor reaction, the user is able to qualitatively capture the measured gelation times of the complex competitor and crosslinking hydrogel system.

To build a more detailed predictive model of gelation time, the kinetic reaction parameters were used to estimate the time required to form the percolation threshold number of crosslinks for any concentration of competitor. Specifically, Matlab was used to numerically solve a system of two coupled-reaction equations: (1) the reaction between the two crosslinking functional groups and (2) the reaction between the competitor and the complementary functional group. The calculated gelation time shows a clear dependence on competitor forward reaction rates and K_{eq} (FIG. 12D). This model also explains why the empirically observed gelation time of HELP is increased with Butanal but is not significantly increased with HydAA. While the addition of HydAA does lead to a modest increase in gelation time, the concentration required to measurably increase it is beyond the scope of concentrations used in this study. Meanwhile, Butanal reacts significantly faster and asymptotically approaches an infinite gelation time, which concurs with the experimental data (20 mM, FIG. 11C,12B) and theory (FIG. 11F).

We propose that by using a competitor to increase gelation times, we can overcome the limitations of rapidly gelling hydrazone-crosslinked HELP. We compared the macroscopic structure of a stiff (˜3.5 kPa) HELP formulation with and without Butanal. Without a competitor, the hydrogel rapidly gelled, resulting in a heterogenous hydrogel with nonuniform distribution of polymer. However, addition of 10 mM Butanal allowed for adequate mixing of the polymers and a homogenous hydrogel (FIG. 12E). To quantitatively evaluate the heterogeneity of HELP hydrogels with or without a competitor, we added a fluorescent dye and compared the spatial distribution of the fluorescent signal. Compared to the rapidly gelling control, the increased gelation time of HELP with Butanal produced a significantly higher distribution of fluorescence intensity, with a significantly lower variance compared to the rapidly gelling condition (FIG. 12F). By using a rapidly reaction competitor to slow down gelation kinetics, we have developed a solution to the challenge of inhomogeneous DCC hydrogels, and produced a uniform HELP hydrogel.

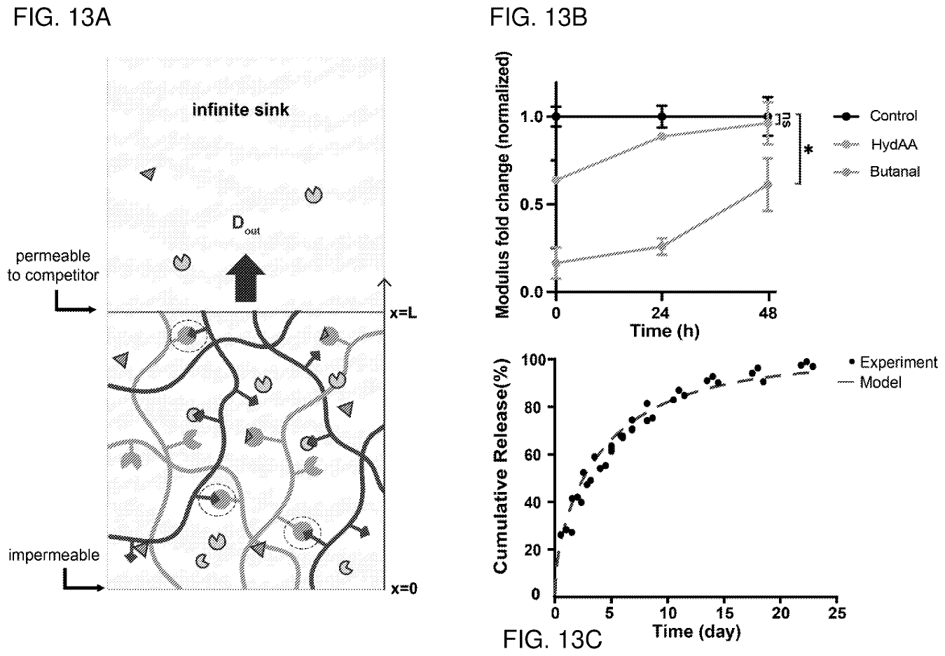

An advantage of using a reversible, small molecule as competitors is the ability for the molecule to diffuse out of the system. After gelation, the competitor can diffuse away from the hydrogel to recover the equilibrium density of crosslinks between the polymers, which results in hydrogel stiffening over time (FIG. 13A). The rate at which competitors diffuse out of the hydrogel depends upon the availability of free, unbound competitors, which is a function of the stability of the hydrazone bond and can be predicted by the K_{eq} of the competitor. Comparing competitors with low and high K_{eq} demonstrates the range of time-scales over which the competitors can diffuse out of the gel. For a low K_{eq} (HydAA), the modulus recovers by 89% over 24 hours and fully recovers after 48 hrs, whereas a higher K_{eq} (Butanal) induces a significantly slower recovery of modulus with 61% at 48 hrs (FIG. 13B). By using a competitor that can delay gelation and diffuse out of the hydrogel, a HELP hydrogel has been produced that is both uniform and stiff.

To predict the rate at which the hydrogel recovers, and the dependence on reaction rates, a reaction-diffusion model of the hydrogel and competitor system was assembled. Taking advantage of the symmetry of the hydrogel, the model was simplified to a 1-dimensional system and the boundary conditions were set by the impermeable culture plate at the bottom of the hydrogel and the infinite sink of competitor-free solution at the top (FIG. 13A). The rate at which the stiffness increases is dependent upon the reaction kinetics and the diffusion rate of the competitor through a system of coupled differential equations. The diffusivity of the freely diffusing small molecule was estimated by a linear extrapolation of diffusion rates of FITC-dextran probes, measured by Fluorescence Recovery After Photobleaching (FRAP).

To experimentally validate this theoretical model, an aldehyde-modified rhodamine (ald-rho) was incorporated into HELP. Using Matlab, we numerically solved the system of equations and compared the predictions with the experimentally observed release profile of ald-rho, which displays a sustained cumulative release over a period of days (FIG. 13C). Both the shape and time-scale of the experimentally observed release profile matched the theoretical predictions. This provides a method by which mechanical properties can be predicted using reaction kinetic parameters of candidate competitors.

An advantage of using a reversible, small molecule as competitors is the ability for the molecule to diffuse out of the system. After gelation, the competitor can diffuse away from the hydrogel to recover the equilibrium density of crosslinks between the polymers, which results in hydrogel stiffening over time (FIG. 13A). The rate at which competitors diffuse out of the hydrogel depends upon the availability of free, unbound competitors, which is a function of the stability of the hydrazone bond and can be predicted by the K_{eq} of the competitor. Comparing competitors with low and high K_{eq} demonstrates the range of time-scales over which the competitors can diffuse out of the gel. For a low K_{eq} (HydAA), the modulus recovers by 89% over 24 hours and fully recovers after 48 hrs, whereas a higher K_{eq} (Butanal) induces a significantly slower recovery of modulus with 61% at 48 hrs (FIG. 13B). By using a competitor that can delay gelation and diffuse out of the hydrogel, a HELP hydrogel that is both uniform and stiff was produced.

To predict the rate at which the hydrogel recovers, and the dependence on reaction rates, a reaction-diffusion model of the hydrogel and competitor system was assembled. Taking advantage of the symmetry of the hydrogel, the model was simplified to a 1-dimensional system and the boundary conditions were set by the impermeable culture plate at the bottom of the hydrogel and the infinite sink of competitor-free solution at the top (FIG. 13A). The rate at which the stiffness increases is dependent upon the reaction kinetics and the diffusion rate of the competitor through a system of coupled differential equations. The diffusivity of the freely diffusing small molecule was estimated by a linear extrapolation of diffusion rates of FITC-dextran probes, measured by Fluorescence Recovery After Photobleaching (FRAP).

To experimentally validate this theoretical model, an aldehyde-modified rhodamine (ald-rho) was incorporated into HELP. Using Matlab, we numerically solved the system of equations and compared the predictions with the experimentally observed release profile of ald-rho, which displays a sustained cumulative release over a period of days (FIG. 13C). Both the shape and time-scale of the experimentally observed release profile matched the theoretical predictions. This provides a method by which mechanical properties can be predicted using reaction kinetic parameters of candidate competitors.

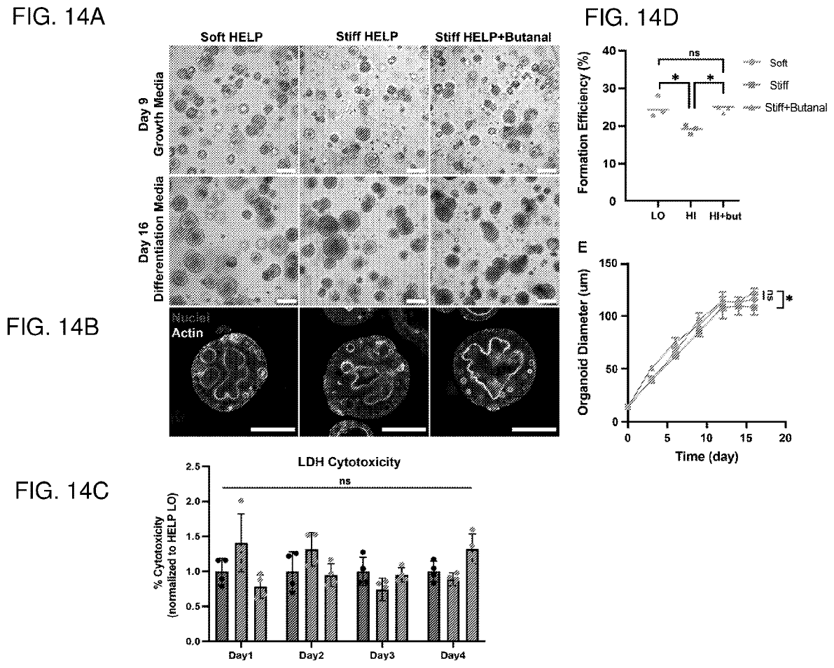

To explore the impact of competitors on the growth and health of cells in culture, a hepatic organoid system was used. Hepatic organoids are especially sensitive to cytotoxic effects as hepatocytes are responsible for xenobiotic metabolism. To explore effects on hepatic organoid growth, HO were cultured as single cells in soft, stiff, and stiff+competitor. A robust formation of HO in the presence of competitor during both the growth and differentiation phase was seen (FIG. 14A). Staining of the cytoskeleton of mature, differentiated HO (Day 16) reveals no aberrant morphology due to the competitor (FIG. 14B). As the competitor diffuses out over several days, with diminishing concentration with time, cytotoxicity was explored during the early phase of cell growth (4-days) when the competitor concentration is highest. Using lactase dehydrogenase, a metric of cytotoxicity, no significant difference in cytotoxicity was observed across all of the conditions (FIG. 14C). Similarly, on day 3, the organoid formation efficiency was quantified, a measure of how many single cells produce organoids. The presence of the competitor led to an increase in formation efficiency compared to the stiff environment (FIG. 14D), and resulted in a formation efficiency was comparable to that of the soft environment. This is mirrored in the organoid growth rate, which shows that over a 16 day culture period, the addition of competitor enabled growth comparable to that of a soft environment (FIG. 14E).

### Methods

Coupled Reaction-Diffusion Equations:

$$\frac{\partial [ A ]}{\partial t} = - {k}_{1} [ A ] [ C ] + {k}_{- 1} [ AC ] - {k}_{2} [ A ] [ B ] + {k}_{- 2} [ AB ] . \qquad 1$$

$$\frac{\partial [ C ]}{\partial t} = D \frac{{\partial }^{2} C}{\partial {x}^{2}} - {k}_{1} [ A ] [ C ] + {k}_{- 1} [ AC ] . \qquad 2$$

$$\frac{\partial [ AC ]}{\partial t} = {k}_{1} [ A ] [ C ] - {k}_{- 1} [ AC ] . \qquad 3$$

$$\frac{\partial [ B ]}{\partial t} = - {k}_{2} [ A ] [ B ] + {k}_{- 2} [ AB ] . \qquad 4$$

$$\frac{\partial [ AB ]}{\partial t} = {k}_{2} [ A ] [ B ] - {k}_{- 1} [ AB ] . \qquad 5$$

Boundary Conditions

At x=0, boundary is impermeable to all species

$$D \frac{\partial [ A ]}{\partial x} {\vert}_{0} = 0. \qquad 1$$

$$D \frac{\partial [ C ]}{\partial x} {\vert}_{0} = 0. \qquad 2$$

$$D \frac{\partial [ AC ]}{\partial x} {\vert}_{0} = 0. \qquad 3$$

$$D \frac{\partial [ B ]}{\partial x} {\vert}_{0} = 0. \qquad 4$$

$$D \frac{\partial [ AB ]}{\partial x} {\vert}_{0} = 0. \qquad 5$$

At x=L, boundary is impermeable to all bound species and permeable to competitor

$$D \frac{\partial [ A ]}{\partial x} {\vert}_{L} = 0. \qquad 1$$

$$[ A ( L , t ) ] = 0. \qquad 2$$

$$D \frac{\partial [ AC ]}{\partial x} {\vert}_{L} = 0. \qquad 3$$

$$D \frac{\partial [ B ]}{\partial x} {\vert}_{L} = 0. \qquad 4$$

$$D \frac{\partial [ AB ]}{\partial x} {\vert}_{L} = 0. \qquad 5$$

Phase Diagram Equations

The equilibrium constant for the competitor,

$${K}_{1} = \frac{[ AC ]}{[ A ] [ C ]} ,$$

and crosslinking,

$${K}_{2} = \frac{[ AB ]}{[ A ] [ B ]}$$

reactions are both dependent upon the concentration of (A). By isolating for species A, the equations can be equated (1), and rearranged as a ratio of the two equilibrium constants (2). D

$$A = \frac{[ AC ]}{[ C ] {K}_{1}} = \frac{[ AB ]}{[ B ] {K}_{2}} \qquad ( 2 )$$

$$\frac{{K}_{1}}{{K}_{2}} = \frac{[ AC ] [ B ]}{[ C ] [ AB ]} \qquad ( 3 )$$

The concentrations of each species can be expressed as the change in the initial concentration, where x₁ and x₂ are the changes in concentration for the competitor and crosslinking reactions, respectively.

$$[ A ] = {[ A ]}_{0} - {x}_{1} - {x}_{2} , [ C ] = {[ C ]}_{0} - {x}_{1} , [ AC ] = {[ AC ]}_{0} + {x}_{1} , \qquad ( 4 )$$

$$[ B ] = {[ B ]}_{0} - {x}_{2} , [ AB ] = {[ AB ]}_{0} + {x}_{2}$$

Substituting the new expressions for concentration into (2), and noting that the initial concentrations of the products, AC and AB, are zero leads to:

$$\frac{{K}_{1}}{{K}_{2}} = \frac{{x}_{1} ( [ {B}_{0} ] - {x}_{2} )}{( [ {C}_{0} ] - {x}_{1} ) {x}_{2}} \qquad ( 5 )$$

Additionally, the extent of reaction of the crosslinking reaction, p, is related to the change in species B (5) and substituted into equation (4).

$$p = \frac{{[ B ]}_{0} - [ B ]}{{[ B ]}_{0}} = \frac{{[ B ]}_{0} - ( {[ B ]}_{0} - {x}_{2} )}{{[ B ]}_{0}} , {x}_{2} = {p [ B ]}_{0} \qquad ( 6 )$$

$$\frac{{K}_{1}}{{K}_{2}} = \frac{{x}_{1} ( {[ B ]}_{0} - {p [ B ]}_{0} )}{( {[ C ]}_{0} - {x}_{1} ) {p [ B ]}_{0}} = \frac{{x}_{1} ( 1 - p )}{( {[ C ]}_{0} - {x}_{1} ) p} \qquad ( 7 )$$

A similar treatment can be applied to the crosslinking reaction (equation 7) to write the change in the competitor reaction, x₁, as a function of the initial concentrations, K₂, and p (equation 8).

$${K}_{2} = \frac{[ AB ]}{[ A ] [ B ]} = \frac{{x}_{2}}{( {[ A ]}_{0} - {x}_{1} - {x}_{2} ) ( {[ B ]}_{0} - x 2 )} = \frac{{p [ B ]}_{0}}{( {[ A ]}_{0} - {x}_{1} - {p [ B ]}_{0} ) ( {[ B ]}_{0} - {p [ B ]}_{0} )} = \frac{p}{( {[ A ]}_{0} - {x}_{1} - {p [ B ]}_{0} ) ( 1 - p )} \qquad ( 8 )$$

$${x}_{1} = {[ A ]}_{0} - \frac{p}{{K}_{2} ( 1 - p )} - {p [ B ]}_{0} \qquad ( 9 )$$

Substituting equation 8 into equation 6 results in equation 9, which can be adjusted to reflect a ratio of the initial concentration of competition and potential crosslinking site, equation 10.

$$\frac{{K}_{1}}{{K}_{2}} = \frac{{x}_{1} ( 1 - p )}{( {[ C ]}_{0} - {x}_{1} ) p} = \frac{( {[ A ]}_{0} - \frac{p}{{K}_{2} ( 1 - p )} - {p [ B ]}_{0} ) ( 1 - p )}{( {[ C ]}_{0} - ( {[ A ]}_{0} - \frac{p}{{K}_{2} ( 1 - p )} - {p [ B ]}_{0} ) ) p} \qquad ( 10 )$$

$$\frac{{K}_{1}}{{K}_{2}} = \frac{( {[ A ]}_{0} - \frac{p}{{K}_{2} ( 1 - p )} - {p [ B ]}_{0} ) ( 1 - p )}{( {R [ A ]}_{0} - ( {[ A ]}_{0} - \frac{p}{{K}_{2} ( 1 - p )} - {p [ B ]}_{0} ) ) p} , where R = \frac{{[ C ]}_{0}}{{[ A ]}_{0}} \qquad ( 11 )$$

Importantly, equation 10 is a function of defined input parameters, including initial concentrations and crosslinking reactions, and is a function of p, the extent of reaction of the crosslinking reaction. The extent of the crosslinking reaction is related to the mean-field theory of gelation, which specifies that a percolated network, i.e., a gel, is formed at a critical extent of reaction, p_{c}, related to the functionality of the polymer, f.

$${p}_{c} = \frac{1}{f - 1} . \qquad 1$$

To find 0.98 limit

$$\frac{{K}_{1}}{{K}_{2}} = \frac{( {[ A ]}_{0} - \frac{p}{{K}_{2} ( 1 - p )} - {p [ B ]}_{0} ) ( 1 - p )}{( {R [ A ]}_{0} - ( {[ A ]}_{0} - \frac{p}{{K}_{2} ( 1 - p )} - {p [ B ]}_{0} ) ) p} rearrange for R , \qquad 1$$

$$R = \frac{1}{{[ A ]}_{0}} ( \frac{{K}_{2}}{{K}_{1} p} ( [ {[ A ]}_{0} - \frac{p}{{K}_{2} ( 1 - p )} - {p [ B ]}_{0} ) ( 1 - p ) + ( {[ A ]}_{0} - \frac{p}{{K}_{2} ( 1 - p )} - {p [ B ]}_{0} ) ) .$$

$$R = \frac{1}{{[ A ]}_{0}} ( \frac{{K}_{2}}{{K}_{1} p} ( {[ A ]}_{0} - \frac{p}{{K}_{2} ( 1 - p )} - {p [ B ]}_{0} ) ( 1 - p ) + ( {[ A ]}_{0} - \frac{p}{{K}_{2} ( 1 - p )} - {p [ B ]}_{0} ) ) - \frac{1}{{[ A ]}_{0}} ( ( {[ A ]}_{0} - \frac{p}{{K}_{2} ( 1 - p )} - {p [ B ]}_{0} ) ) = 0.98 . \qquad 2$$

### REFERENCES

Burdick, J. A. and G. D. Prestwich (2011). “Hyaluronic Acid Hydrogels for Biomedical Applications.” Advanced Materials 23(12): H41-H56.
Burdick, J. A. and M. M. Stevens (2005). Biomedical hydrogels. Biomaterials, Artificial Organs and Tissue Engineering: 107-115.
Chaudhuri, O., L. Gu, M. Darnell, D. Klumpers, S. A. Bencherif, J. C. Weaver, N. Huebsch and D. J. Mooney (2015). “Substrate stress relaxation regulates cell spreading.” Nat Commun 6:6364.
Chaudhuri, O., L. Gu, D. Klumpers, M. Darnell, S. A. Bencherif, J. C. Weaver, N. Huebsch, H. P. Lee, E. Lippens, G. N. Duda and D. J. Mooney (2016). “Hydrogels with tunable stress relaxation regulate stem cell fate and activity.” Nat Mater 15(3): 326-334.
De Gennes, P. G. (1976). “On a relation between percolation theory and the elasticity of gels.” Journal de Physique Lettres 37(1): 1-2.
Dicker, K. T., L. A. Gurski, S. Pradhan-Bhatt, R. L. Witt, M. C. Farach-Carson and X. Jia (2014). “Hyaluronan: a simple polysaccharide with diverse biological functions.” Acta Biomater 10(4): 1558-1570.
Dirksen, A., S. Dirksen, T. M. Hackeng and P. E. Dawson (2006). “Nucleophilic catalysis of hydrazone formation and transimination: implications for dynamic covalent chemistry.” J Am Chem Soc 128(49): 15602-15603.
Drury, J. L. and D. J. Mooney (2003). “Hydrogels for tissue engineering: scaffold design variables and applications.” Biomaterials 24(24): 4337-4351.
Gilchrist, A. E. and B. A. C. Harley (2021). “Engineered Tissue Models to Replicate Dynamic Interactions within the Hematopoietic Stem Cell Niche.” Adv Healthc Mater: e2102130.
Gilchrist, A. E., S. Lee, Y. Hu and B. A. C. Harley (2019). “Soluble Signals and Remodeling in a Synthetic Gelatin-Based Hematopoietic Stem Cell Niche.” Adv Healthc Mater 8(20): e1900751.
Gilchrist, A. E., J. F. Serrano, M. T. Ngo, Z. Hrnjak, S. Kim and B. A. C. Harley (2021). “Encapsulation of murine hematopoietic stem and progenitor cells in a thiol-crosslinked maleimide-functionalized gelatin hydrogel.” Acta Biomater 131: 138-148.
Ho, S. S., A. T. Keown, B. Addison and J. K. Leach (2017). “Cell Migration and Bone Formation from Mesenchymal Stem Cell Spheroids in Alginate Hydrogels Are Regulated by Adhesive Ligand Density.” Biomacromolecules 18(12): 4331-4340.
Hu, W., Z. Wang, Y. Xiao, S. Zhang and J. Wang (2019). “Advances in crosslinking strategies of biomedical hydrogels.” Biomater Sci 7(3): 843-855.
Hull, S. M., J. Lou, C. D. Lindsay, R. S. Navarro, B. Cai, L. G. Brunel, A. D. Westerfield, Y. Xia and S. C. Heilshorn (2023). “3D bioprinting of dynamic hydrogel bioinks enabled by small molecule modulators.” Science Advances 9(13).
Hunt, D. R., K. C. Klett, S. Mascharak, H. Wang, D. Gong, J. Lou, X. Li, P. C. Cai, R. A. Suhar, J. Y. Co, B. L. LeSavage, A. A. Foster, Y. Guan, M. R. Amieva, G. Peltz, Y. Xia, C. J. Kuo and S. C. Heilshorn (2021). “Engineered Matrices Enable the Culture of Human Patient-Derived Intestinal Organoids.” Adv Sci (Weinh) 8(10): 2004705.
Jencks, W. P. (2007). Mechanism and Catalysis of Simple Carbonyl Group Reactions. Progress in Physical Organic Chemistry: 63-128.
Kalia, J. and R. T. Raines (2008). “Hydrolytic Stability of Hydrazones and Oximes.” Angewandte Chemie International Edition 47(39): 7523-7526.
Kolmel, D. K. and E. T. Kool (2017). “Oximes and Hydrazones in Bioconjugation: Mechanism and Catalysis.” Chem Rev 117(15): 10358-10376.
Kool, E. T., D. H. Park and P. Crisalli (2013). “Fast hydrazone reactants: electronic and acid/base effects strongly influence rate at biological pH.” J Am Chem Soc 135(47): 17663-17666.
Lee, K. Y. and D. J. Mooney (2001). “Hydrogels for tissue engineering.” Chem Rev 101(7): 1869-1879.
LeSavage, B. L., A. E. Gilchrist, B. A. Krajina, K. Karlsson, A. R. Smith, K. Karagyozova, K. C. Klett, C. Curtis, C. J. Kuo and S. C. Heilshorn (2022). “Engineered extracellular matrices reveal stiffness-mediated chemoresistance in patient-derived pancreatic cancer organoids.” BioRxiv.
LeSavage, B. L., N. A. Suhar, C. M. Madl and S. C. Heilshorn (2018). “Production of Elastin-like Protein Hydrogels for Encapsulation and Immunostaining of Cells in 3D.” J Vis Exp(135).
Lou, J., R. Stowers, S. Nam, Y. Xia and O. Chaudhuri (2018). “Stress relaxing hyaluronic acid-collagen hydrogels promote cell spreading, fiber remodeling, and focal adhesion formation in 3D cell culture.” Biomaterials 154: 213-222.
Lou, J. and Y. Xia (2022). “Using Competitor Molecules to Reversibly Modulate the Mechanical Properties of Viscoelastic Hydrogels.” ACS Macro Lett 11(11): 1312-1316.
Madl, C. M. and S. C. Heilshorn (2019). “Rapid Diels-Alder Cross-linking of Cell Encapsulating Hydrogels.” Chemistry of Materials 31(19): 8035-8043.
Madl, C. M., L. M. Katz and S. C. Heilshorn (2016). “Bio-Orthogonally Crosslinked, Engineered Protein Hydrogels with Tunable Mechanics and Biochemistry for Cell Encapsulation.” Adv Funct Mater 26(21): 3612-3620.
Mckinnon, D. D., D. W. Domaille, J. N. Cha and K. S. Anseth (2014). “Biophysically Defined and Cytocompatible Covalently Adaptable Networks as Viscoelastic 3D Cell Culture Systems.” Advanced Materials 26(6): 865-872.
Mckinnon, D. D., D. W. Domaille, J. N. Cha and K. S. Anseth (2014). “Biophysically defined and cytocompatible covalently adaptable networks as viscoelastic 3D cell culture systems.” Adv Mater 26(6): 865-872.
Muir, V. G. and J. A. Burdick (2021). “Chemically Modified Biopolymers for the Formation of Biomedical Hydrogels.” Chem Rev 121(18): 10908-10949.
Navarro, R. S., M. S. Huang, J. G. Roth, K. M. Hubka, C. M. Long, A. Enejder and S. C. Heilshorn (2022). “Tuning Polymer Hydrophilicity to Regulate Gel Mechanics and Encapsulated Cell Morphology.” Adv Healthc Mater 11(13): e2200011.
Peppas, N. A., J. Z. Hilt, A. Khademhosseini and R. Langer (2006). “Hydrogels in Biology and Medicine: From Molecular Principles to Bionanotechnology.” Advanced Materials 18(11): 1345-1360.
Raeber, G. P., M. P. Lutolf and J. A. Hubbell (2005). “Molecularly engineered PEG hydrogels: a novel model system for proteolytically mediated cell migration.” Biophys J 89(2): 1374-1388.
Rizwan, M., A. E. G. Baker and M. S. Shoichet (2021). “Designing Hydrogels for 3D Cell Culture Using Dynamic Covalent Crosslinking.” Adv Healthc Mater 10(12): e2100234.
Rosales, A. M. and K. S. Anseth (2016). “The design of reversible hydrogels to capture extracellular matrix dynamics.” Nat Rev Mater 1.
Rowan, S. J., S. J. Cantrill, G. R. L. Cousins, J. K. M. Sanders and J. F. Stoddart (2002). “Dynamic Covalent Chemistry.” Angewandte Chemie International Edition 41(6): 898-952.
Rubinstein, M. and R. H. Colby (2003). Polymer Physics, Oxford University Press: 199-252.
Saito, F., H. Noda and J. W. Bode (2015). “Critical evaluation and rate constants of chemoselective ligation reactions for stoichiometric conjugations in water.” ACS Chem Biol 10(4): 1026-1033.
Saraswathibhatla, A., D. Indana and O. Chaudhuri (2023). “Cell-extracellular matrix mechanotransduction in 3D.” Nat Rev Mol Cell Biol.
Shayan, M., M. S. Huang, R. Navarro, G. Chiang, C. Hu, B. P. Oropeza, P. K. Johansson, R. A. Suhar, A. A. Foster, B. L. LeSavage, M. Zamani, A. Enejder, J. G. Roth, S. C. Heilshorn and N. F. Huang (2023). “Elastin-like protein hydrogels with controllable stress relaxation rate and stiffness modulate endothelial cell function.” J Biomed Mater Res A.
Steele, A. N., L. M. Stapleton, J. M. Farry, H. J. Lucian, M. J. Paulsen, A. Eskandari, C. E. Hironaka, A. D. Thakore, H. Wang, A. C. Yu, D. Chan, E. A. Appel and Y. J. Woo (2019). “A Biocompatible Therapeutic Catheter-Deliverable Hydrogel for In Situ Tissue Engineering.” Advanced Healthcare Materials 8(5): 1801147.
Stevens, K. R., J. S. Miller, B. L. Blakely, C. S. Chen and S. N. Bhatia (2015). “Degradable hydrogels derived from PEG-diacrylamide for hepatic tissue engineering.” J Biomed Mater Res A 103(10): 3331-3338.
Suhar, R. A., V. M. Doulames, Y. Liu, M. E. Hefferon, O. Figueroa, 3rd, H. Buabbas and S. C. Heilshorn (2022). “Hyaluronan and elastin-like protein (HELP) gels significantly improve microsphere retention in the myocardium.” Biomater Sci 10(10): 2590-2608.
Tang, S., B. M. Richardson and K. S. Anseth (2021). “Dynamic covalent hydrogels as biomaterials to mimic the viscoelasticity of soft tissues.” Progress in Materials Science 120.
Teng, L., Y. Chen, Y. G. Jia and L. Ren (2019). “Supramolecular and dynamic covalent hydrogel scaffolds: from gelation chemistry to enhanced cell retention and cartilage regeneration.” J Mater Chem B 7(43): 6705-6736.
Wang, H., L. Cai, A. Paul, A. Enejder and S. C. Heilshorn (2014). “Hybrid elastin-like polypeptide-polyethylene glycol (ELP-PEG) hydrogels with improved transparency and independent control of matrix mechanics and cell ligand density.” Biomacromolecules 15(9): 3421-3428.
Wang, H. and S. C. Heilshorn (2015). “Adaptable Hydrogel Networks with Reversible Linkages for Tissue Engineering.” Advanced Materials 27(25): 3717-3736.
Wang, H., D. Zhu, A. Paul, L. Cai, A. Enejder, F. Yang and S. C. Heilshorn (2017). “Covalently adaptable elastin-like protein-hyaluronic acid (ELP-HA) hybrid hydrogels with secondary thermoresponsive crosslinking for injectable stem cell delivery.” Adv Funct Mater 27(28).
Wang, L. L., C. B. Highley, Y. C. Yeh, J. H. Galarraga, S. Uman and J. A. Burdick (2018). “Three-dimensional extrusion bioprinting of single- and double-network hydrogels containing dynamic covalent crosslinks.” J Biomed Mater Res A 106(4): 865-875.
Winne, J. M., L. Leibler and F. E. Du Prez (2019). “Dynamic covalent chemistry in polymer networks: a mechanistic perspective.” Polymer Chemistry 10(45): 6091-6108.
Wong Po Foo, C. T., J. S. Lee, W. Mulyasasmita, A. Parisi-Amon and S. C. Heilshorn (2009). “Two-component protein-engineered physical hydrogels for cell encapsulation.” Proc Natl Acad Sci USA 106(52): 22067-22072.
Zhu, D., H. Wang, P. Trinh, S. C. Heilshorn and F. Yang (2017). “Elastin-like protein-hyaluronic acid (ELP-HA) hydrogels with decoupled mechanical and biochemical cues for cartilage regeneration.” Biomaterials 127:132-140.

1.-34. (canceled)

35. A method of culturing cells in a 3D cell culture, the method comprising:
encapsulating an initiating population of mammalian cells in the hydrogel solution comprising:
a hydrogel solution polymerized from a mixture of a first polymer modified with a first functional group and a second polymer modified with a second functional group, wherein the first functional group participates in reversible covalent bond formation with the second functional group, and
contacting the encapsulated mammalian cells with a competitor agent that competes for binding between the first and second functional group, in an amount sufficient to reduce the stiffness of the hydrogel, and
maintaining the encapsulated cells in suitable medium.

36. The method of claim 35, further comprises contacting the hydrogel solution with an effective amount of a catalyst agent that alters the forward and reverse reaction rate of the reversible covalent bond formation in the hydrogel solution.

37. The method of claim 35, wherein the first polymer comprises hyaluronic acid and second polymer comprises elastin-like protein.

38. The method of claim 35, wherein the first functional group comprises aldehyde or benzaldehyde and the second functional group comprises hydrazine.

39. The method of claim 35, wherein the first functional group comprises hydrazine and the second functional group comprises aldehyde or benzaldehyde.

40. The method of claim 35, wherein the competitor agent is selected from the group of 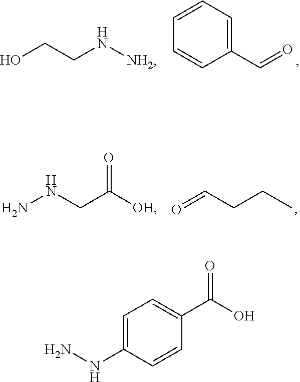
and derivatives thereof.

41. The method of claim 35, wherein the catalyst agent is

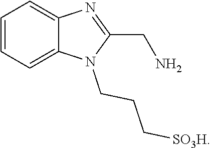 [SMILES: NCc1nc2ccccc2n1CCCS(=O)(=O)O]

42. The method of claim 36, wherein the catalyst agent is at a concentration of from about 5 mM to about 25 mM.

43. The method of claim 35, wherein the initiating cell population comprises a single cell suspension.

45. The method of claim 35, wherein the cell population comprises stem cells.

46. The method of claim 35, wherein the initiating cell population comprises a tissue explant.

47. The method of claim 35, wherein the initiating cell population differentiates into organoids in culture.

48. The method of claim 35, wherein the initiating cell population comprises hepatic cells.

49. A composition comprising:
a hydrogel solution of hydrogel polymerized therefrom comprising a mixture of a first polymer modified with a first functional group and a second polymer modified with a second functional group, wherein the first functional group participates in reversible covalent bond formation with the second functional group,
and a competitor agent that competes for binding between the first and second functional group, in an amount effective to modulate the stiffness of the hydrogel polymerized from the hydrogel solution.

50. The composition of claim 49, wherein the competitor agent is selected from the group of 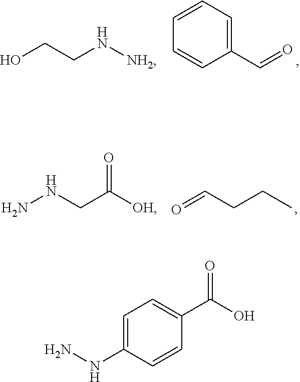
and derivatives thereof.

51. The composition of claim 49, further comprises a catalyst agent that is

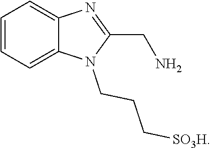 [SMILES: NCc1nc2ccccc2n1CCCS(=O)(=O)O]

52. The composition of claim 49, comprising viable cells for culture.


SEQUENCE LISTING (28 sequences)

SEQ ID NO: 1 - protein, 378 aa, synthetic construct
  REGION 1..378: note = synthetic sequence
LQLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRGDSPASSAVPGIGVPGIGVPGKGVPGIGVPGIGVPGIGVPGIGVPGKGVPGIGVPGIGVPGIGVPGIGVPGKGVPGIGVPGIG

SEQ ID NO: 2 - protein, 378 aa, synthetic construct
  REGION 1..378: note = synthetic sequence
LQLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIGLDASTVYAVGRDGSPASSAVPGIGVPGIGVPGKGVPGIGVPGIGVPGIGVPGIGVPGKGVPGIGVPGIGVPGIGVPGIGVPGKGVPGIGVPGIG

SEQ ID NO: 3 - protein, 4 aa, synthetic construct
  REGION 1..4: note = synthetic sequence
DGEA

SEQ ID NO: 4 - protein, 12 aa, synthetic construct
  REGION 1..12: note = synthetic sequence
PLAEIDGIELTY

SEQ ID NO: 5 - protein, 8 aa, synthetic construct
  REGION 1..8: note = synthetic sequence
VFDNFVLK

SEQ ID NO: 6 - protein, 5 aa, synthetic construct
  REGION 1..5: note = synthetic sequence
IKVAV

SEQ ID NO: 7 - protein, 5 aa, synthetic construct
  REGION 1..5: note = synthetic sequence
YIGSR

SEQ ID NO: 8 - protein, 5 aa, synthetic construct
  REGION 1..5: note = synthetic sequence
HAVDI

SEQ ID NO: 9 - protein, 10 aa, synthetic construct
  REGION 1..10: note = synthetic sequence
HAVDIHAVDI

SEQ ID NO: 10 - protein, 17 aa, synthetic construct
  REGION 1..17: note = synthetic sequence
TVYAVTGRGDSPASSAA

SEQ ID NO: 11 - protein, 20 aa, synthetic construct
  REGION 1..20: note = synthetic sequence
VSDPGYIGSRSDDSASASAA

SEQ ID NO: 12 - protein, 16 aa, synthetic construct
  REGION 1..16: note = synthetic sequence
VGPAGGDGEAGAQGPP

SEQ ID NO: 13 - protein, 28 aa, synthetic construct
  REGION 1..28: note = synthetic sequence
SGSGGSGGPLAEIDGIELTYGGSGGSGS

SEQ ID NO: 14 - protein, 26 aa, synthetic construct
  REGION 1..26: note = synthetic sequence
SGSGGSGGLDVFDNFVLKGGSGGSGS

SEQ ID NO: 15 - protein, 25 aa, synthetic construct
  REGION 1..25: note = synthetic sequence
SGSGGSGGLDVFDNFVLGGSGGSGS

SEQ ID NO: 16 - protein, 21 aa, synthetic construct
  REGION 1..21: note = synthetic sequence
SGSGGSGGHAVDIGGSGGSGS

SEQ ID NO: 17 - protein, 28 aa, synthetic construct
  REGION 1..28: note = synthetic sequence
SGSGGSGGHAVDINGHAVDIGGSGGSGS

SEQ ID NO: 18 - protein, 21 aa, synthetic construct
  REGION 1..21: note = synthetic sequence
SGSGGSGGADHIVGGSGGSGS

SEQ ID NO: 19 - protein, 17 aa, synthetic construct
  REGION 1..17: note = synthetic sequence
TVYAVTGRDGSPASSAA

SEQ ID NO: 20 - protein, 25 aa, synthetic construct
  REGION 1..25: note = synthetic sequence
VPGIGVPGIGVPGKGVPGIGVPGIG

SEQ ID NO: 21 - protein, 30 aa, synthetic construct
  REGION 1..30: note = synthetic sequence
VPGIGVPGKGVPGYGVPGIGVPGKGVPGIG

SEQ ID NO: 22 - protein, 19 aa, synthetic construct
  REGION 1..19: note = synthetic sequence
ARKQAASIKVAVSADRASA

SEQ ID NO: 23 - protein, 5 aa, synthetic construct
  REGION 1..5: note = synthetic sequence
VPGIG

SEQ ID NO: 24 - protein, 5 aa, synthetic construct
  REGION 1..5: note = synthetic sequence
VPGKG

SEQ ID NO: 25 - protein, 5 aa, synthetic construct
  REGION 1..5: note = synthetic sequence
VPGYG

SEQ ID NO: 26 - protein, 5 aa, synthetic construct
  REGION 1..5: note = synthetic sequence
GGGGS

SEQ ID NO: 27 - protein, 14 aa, synthetic construct
  REGION 1..14: note = synthetic sequence
GSTSGSGKSSEGKG

SEQ ID NO: 28 - protein, 360 aa, synthetic construct
TVYAVGRGDSPASSAVPGIGVPGIGVPGKGVPGIGVPGIGVPGIGVPGIGVPGKGVPGIGVPGIGVPGIGVPGIGVPGKGVPGIGVPGIGTVYAVGRGDSPASSAVPGIGVPGIGVPGKGVPGIGVPGIGVPGIGVPGIGVPGKGVPGIGVPGIGVPGIGVPGIGVPGKGVPGIGVPGIGTVYAVGRGDSPASSAVPGIGVPGIGVPGKGVPGIGVPGIGVPGIGVPGIGVPGKGVPGIGVPGIGVPGIGVPGIGVPGKGVPGIGVPGIGTVYAVGRGDSPASSAVPGIGVPGIGVPGKGVPGIGVPGIGVPGIGVPGIGVPGKGVPGIGVPGIGVPGIGVPGIGVPGKGVPGIGVPGIG